(19) United States
(12) Patent Application Publication
(10) Pub. No.: US 2005/0282251 A1
(43) Pub. Date: Dec. 22, 2005

(54) Heparanase II, a novel human heparanase paralog

(75) Inventors: Robert Leroy Heinrikson, Plainwell, MI (US); Michael Jerome Bienkowski, Portage, MI (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 11/057,732

(22) Filed: Feb. 14, 2005

Related U.S. Application Data
(63) Continuation of application No. 09/836,461, filed on Apr. 17, 2001, now abandoned.
(60) Provisional application No. 60/199,072, filed on Apr. 20, 2000.

(51) Int. Cl. C07H 21/04; C12N 9/24; C07K 16/40; C12P 21/06; C12N 15/09

(52) U.S. Cl. 435/69.1; 435/200; 435/320.1; 435/325; 530/388.26; 536/23.2

(57) ABSTRACT

The present invention provides a cDNA encoding a heretofore unknown enzyme termed heparanase II; constructs and recombinant host cells incorporating the cDNA; the heparanase II polypeptide encoded by the gene; antibodies to the polypeptide; and methods of making and using all of the foregoing.

This application claims the benefit of U.S. provisional application Ser. No. 60/199,072 filed Apr. 20, 2000, and U.S. Ser. No. 09/836,461 filed Apr. 17, 2001 under 35 USC § 119(e)(i).

### BACKGROUND OF THE INVENTION

1. Field of the Invention

The present invention provides isolated heparanase II polypeptides, and the isolated polynucleotide molecules that encode them, as well as vectors and host cells comprising such polynucleotide molecules. The invention also provides a method for the identification of an agent that alters heparanase activity.

2. Description of Related Art

Heparanase is an enzyme that can degrade both heparin proteoglycans (HPG) and heparan sulfate proteoglycans (HSPG). Heparanase activity in mammalian cells is well known. The activity has been identified in various melanoma cells (Nakajima, et al., Cancer Letters 31: 277-283, 1986), mammary adenocarcinoma cells (Parish, et al., Int. J. Cancer, 40: 511-518, 1987), leukemic cells (Yahalom, et al., Leukemia Research 12: 711-717, 1988), prostate carcinoma cells (Kosir, et al., J. Surg. Res. 67: 98-105, 1997), mast cells (Ogren and Lindahl, J. Biol. Chem. 250: 2690-2697, 1975), macrophages (Savion, et al., J. Cell. Physiol., 130: 85-92, 1987), mononuclear cells (Sewell, et al., Biochem. J. 264: 777-783, 1989), neutrophils (Matzner, et al. 51: 519-524, 1992, T-cells (Vettel et al., Eur J. Immunol. 21: 2247-2251, 1991), platelets (Haimovitz-Friedman, et al., Blood 78: 789-796, 1991), endothelial cells (Godder, et al., J. Cell Physiol. 148: 274-280, 1991), and placenta (Klein and von Figura, BBRC 73: 569, 1976). An earlier report that human platelet heparanase is a member of the CXC chemokine family (Hoogewerf et al., J. Biol. Chem. 270: 3268-3277) is controversial.

Elevated heparanase activity has been documented in mobile, invasive cells. Examples include invasive melanoma, lymphoma, mastocytoma, mammary adeno-carcinoma, leukemia, and rheumatoid fibroblasts. Heparanase activity has also been documented in non-pathologic situations involving the migration of lymphocytes, neutrophils, macrophages, eosinophils and platelets (Vlodavsky et al., Invasion Metastasis 12: 112-127, 1992).

In view of the observation that heparanase activity is present in mobile, invasive cells associated with pathologic states, it may be hypothesized that an inhibitor of heparanase would broadly influence the invasive potential of these diverse cells. Further, inhibition of heparan sulfate degradation would inhibit the release of bound growth factors and other biologic response modifiers that would, if released, fuel the growth of adjacent tissues and provide a supportive environment for cell growth (Rapraeger et al., Science 252: 1705-1708, 1991). Inhibitors of heparanase activity would also be of value in the treatment of arthritis, asthma, and other inflammatory diseases, vascular restenosis, arteriosclerosis, tumor growth and progression, and fibro-proliferative disorders.

Because heparanase breaks down the extracellular matrix with attendant release of growth factors, enzymes, and chemotactic proteins, an agent that inhibits heparanase activity should find therapeutic application in cancer, CNS and neurodegenerative diseases, inflammation, and in cardiovascular diseases such as restenosis following angioplasty and arteriosclerosis. A major obstacle to designing a screening assay for the identification of inhibitors of mammalian heparanase activity has been the difficulty of purifying any mammalian heparanase to homogeneity so as to determine its structure, including its amino acid sequence. For this reason, therapeutic applications of mammalian heparanase, or of inhibitors of mammalian heparanase, have been based on research carried out using bacterial heparanase.

Heparanases themselves are useful for a variety of purposes. These applications include, the acceleration of wound healing, the blocking of angiogenesis, and the degradation of heparin and the neutralization of heparin's anticoagulant properties during surgery, wherein an immobilized heparanase filter is connected to extracorporeal devices to degrade heparin and neutralize its anticoagulant properties during surgery. Immobilization onto filters can be achieved by methods well known in the art, such as those disclosed by Langer et a/. (Biomaterials: Inter-facial Phenomenon and Applications, Cooper et al., eds., pp. 493-509 (1982)), and in U.S. Pat. Nos. 4,373,023, 4,863,611 and 5,211,850.

WO 91/02977 describes a substantially, but partially, purified heparanase produced by cation exchange resin chromatography and the affinity absorbent purification of heparanase-containing extract from the human SK-HEP-1 cell line. WO 91/02977 also describes a method of promoting wound healing utilizing compositions comprising a “purified” form of heparanase. This enzyme was not thoroughly characterized, and its amino acid sequence was not determined. WO 98/03638 describes a method for the purification of mammalian heparanase from a heparanase-containing material, such as human platelets. However, the amino acid sequence of this heparanase, and the sequence of the polynucleotide molecule that encodes it, are not disclosed in this reference. Furthermore, this heparanase is characterized only as having a native molecular mass of about 50 kDa, and as degrading both heparin and heparan sulfate. The amino acid and nucleic acid sequences of a human heparanase I have been disclosed by Fairbanks et al. and Vlodavsky et al. The sequences of Fairbanks and Vladovsky however, are considerably different than those disclosed here. The sequences are compared in FIG. 2.

In view of the foregoing, it will be clear that there is a need in the art for recombinantly produced human heparanase and that to the extent that multiple heparanase activities are present within the mammalian species that the cloning, isolation and expression of the molecular species responsible for such activities serves a valuable function.

### REFERENCES CITED

U.S. Patent Documents
1. U.S. Pat. No. 4,373,023, Langer et al., Process for neutralizing heparin
2. U.S. Pat. No. 4,863,611, Bernstein et al., Extracorporeal reactors containing immobilized species
3. U.S. Pat. No. 5,211,850, Shettigar et al. Plasma filter sorbent system for removal of components from blood
4. U.S. Pat. No. 5,567,417, Method for inhibiting angiogenesis using heparinase

Patent Documents
1. WO 91/02977, Wound Healing Preparations Containing Heparanase
2. WO 98/03638, Detection of Mammalian Heparanase Activity and Purification of Mammalian Heparanase
3. WO 97/11684, Use of Heparanase to Decrease Inflammatory Responses
4. EP-A-0367566; and WO 91/18982, Type II Interleukin-1 Receptors
5. WO 94/12650, Activating Expression of an Amplifying Endogenous Gene by Homologous Recombination
6. WO 92/20808, Genomic Modifications with Homologous DNA Targeting
7. WO 91/09955, Endogenous Gene Expression Modification with Regulatory Element
8. WO 97/09433, Cell-Cycle Checkpoint Genes
9. W093/11236, Production of Anti-Self Antibodies From Antibody Segment Repertoires and Displayed on Phage

Non-Patent Documents
1. Anderson, W. F., Human gene therapy. Nature, 1998. 392(6679 Suppl): p. 25-30.
2. Aujame, L., F. Geoffroy, and R. Sodoyer, High affinity human antibodies by phage display. Hum Antibodies, 1997. 8(4): p. 155-68.
3. Bruggemann, M. and M. S. Neuberger, Strategies for expressing human antibody repertoires in transgenic mice. Immunol Today, 1996. 17(8): p. 391-7.
4. Bruggemann, M. and M. J. Taussig, Production of human antibody repertoires in transgenic mice. Curr Opin Biotechnol, 1997. 8(4): p. 455-8.
5. Capecchi, M. R., Altering the genome by homologous recombination. Science, 1989. 244(4910): p. 1288-92.
6. Choo, Y., et al., Promoter-specific activation of gene expression directed by bacteriophage-selected zinc fingers. J Mol Biol, 1997. 273(3): p. 525-32.
7. Cosman, D., et al., Cloning, sequence and expression of human interleukin-2 receptor. Nature, 1984. 312(5996): p. 768-71.
8. Cosman, D., et al., High level stable expression of human interleukin-2 receptors in mouse cells generates only low affinity interleukin-2 binding sites. Mol Immunol, 1986. 23(9): p. 935-41.
9. Dayhoff, in Atlas of Protein Sequence and Structure, Vol. 5, p. 124, National Biochemical Research Foundation, Washington, D.C. (1972)
10. Erlich H. A., ed., PCR Technology, Stockton Press, New York, 1989;
11. Foote, J. and G. Winter, Antibody framework residues affecting the conformation of the hypervariable loops. J Mol Biol, 1992. 224(2): p. 487-99.
12. Gluzman, Y., SV40-transformed simian cells support the replication of early SV40 mutants. Cell, 1981. 23(1): p. 175-82.
13. Godder, K., et al., Heparanase activity in cultured endothelial cells. J Cell Physiol, 1991. 148(2): p. 274-80.
14. Greisman, H. A. and C. O. Pabo, A general strategy for selecting high-affinity zinc finger proteins for diverse DNA target sites. Science, 1997. 275(5300): p. 657-61.
15. Haimovitz-Friedman, A., et al., Activation of platelet heparitinase by tumor cell-derived factors. Blood, 1991. 78(3): p. 789-96.
16. Harlow et al. (Eds), Antibodies A Laboratory Manual; Cold Spring Harbor Laboratory; Cold Spring Harbor, N.Y. (1988), Chapter 6
17. Fairbanks M B et al Processing of the human heparanase precursor and evidence that the active enzyme is a heterodimer. 1999 Journal of Biological Chemistry. 274(42):29587-90,
18. Hulett, M. D., et al., Cloning of mammalian heparanase, an important enzyme in tumor invasion and metastisis. Nature Genetics, 1999 5:803-809
19. Hoogenboom, H. R., Designing and optimizing library selection strategies for generating high-affinity antibodies. Trends Biotechnol, 1997. 15(2): p. 62-70.
20. Hoogewerf, A. J., et al., CXC chemokines connective tissue activating peptide-III and neutrophil activating peptide-2 are heparin/heparan sulfate-degrading enzymes. J Biol Chem, 1995. 270(7): p. 3268-77.
21 Hopp, T. P. and K. R. Woods, A computer program for predicting protein antigenic determinants. Mol Immunol, 1983. 20(4): p. 483-9.
22. Hopp, T. P. and K. R. Woods, Prediction of protein antigenic determinants from amino acid sequences. Proc Natl Acad Sci USA, 1981. 78(6): p. 3824-8.
23. Innis M. A., et al. eds PCR Protocols: A Guide to Methods and Applications 1990, Academic Press, Inc., New York,
24. Jones, P. T., et al., Replacing the complementarity-determining regions in a human antibody with those from a mouse. Nature, 1986. 321(6069): p. 522-5.
25. Kettleborough, C. A., et al, Humanization of a mouse monoclonal antibody by CDR-grafting: the importance of framework residues on loop conformation. Protein Eng, 1991. 4(7): p. 773-83.
26. Khan, M. Y. and S. A. Newman, A rapid colorimetric assay for heparinase activity. Anal Biochem, 1991. 196(2): p. 373-6.
27. Kim, J. S., et al., Design of TATA box-binding protein/zinc finger fusions for targeted regulation of gene expression. Proc Natl Acad Sci USA, 1997. 94(8): p. 3616-20.
28. Klein, U. and K. Von Figura, Partial purification and characterization of heparan sulfate specific endoglucuronidase. Biochem Biophys Res Commun, 1976. 73(3): p. 569-76.
29. Kosir, M. A., et al., Human prostate carcinoma cells produce extracellular heparanase. J Surg Res, 1997. 67(1): p. 98-105.
30. Kussie, P. H. et al. Cloning and Functional Expression of a Human Heparanase Gene, Biochem. Biophys. Res. Comm. 1999 261:183-187.
31. Langer et al. Biomaterials: Inter-facial Phenomenon and Applications, Cooper et al., eds., pp. 493-509 (1982)
32. Lehninger, Biochemistry, Second Edition; Worth Publishers, Inc. NY:N.Y. (1975), pp.71-77
33. Liu, Q., et al., Design of polydactyl zinc-finger proteins for unique addressing within complex genomes. Proc Natl Acad Sci USA, 1997. 94(11): p. 5525-30.
34. Margalit, H., et al., Prediction of immunodominant helper T cell antigenic sites from the primary sequence. J Immunol, 1987.138(7): p. 2213-29
35. Matzner, Y., et al., Subcellular localization of heparanase in human neutrophils. J Leukoc Biol, 1992. 51(6): p. 519-24.
36. McColl, D. J., C. D. Honchell, and A. D. Frankel, Structure-based design of an RNA-binding zinc finger. Proc Natl Acad Sci USA, 1999. 96(17): p. 9521-6.
37. Miller, A. D., Human gene therapy comes of age. Nature, 1992. 357(6378): p. 455-60.
38. Nakajima, M., T. Irimura, and G. L. Nicolson, Tumor metastasis-associated heparanase (heparan sulfate endoglycosidase) activity in human melanoma cells. Cancer Lett, 1986. 31(3): p. 277-83.
39. Ogren, S. and U. Lindahl, Cleavage of macromolecular heparin by an enzyme from mouse mastocytoma. J Biol Chem, 1975. 250(7): p. 2690-7.
40. Okayama, H. and P. Berg, A cDNA cloning vector that permits expression of cDNA inserts in mammalian cells. Mol Cell Biol, 1983. 3(2): p. 280-9.
41. Parish, C. R., et al., Evidence that sulphated polysaccharides inhibit tumour metastasis by blocking tumour-cell-derived heparanases. Int J Cancer, 1987. 40(4): p. 511-8.
42. Rader, C. and C. F. Barbas, 3rd, Phage display of combinatorial antibody libraries. Curr Opin Biotechnol, 1997. 8(4): p. 503-8.
43. Rapraeger, A. C., A. Krufka, and B. B. Olwin, Requirement of heparan sulfate for bFGF-mediated fibroblast growth and myoblast differentiation. Science, 1991. 252(5013): p. 1705-8.
44. Riechmann, L., et al., Reshaping human antibodies for therapy. Nature, 1988. 332(6162): p. 323-7.
45. Sambrook, et al., (Eds.), Molecular Cloning: A Laboratory Manual, Cold Spring Harbor Laboratory Press: Cold Spring Harbor, N.Y. (1989).
46. Savion, N., M. H. Disatnik, and Z. Nevo, Murine macrophage heparanase: inhibition and comparison with metastatic tumor cells. J Cell Physiol, 1987. 130(1): p. 77-84.
47. Segal, D. J., et al., Toward controlling gene expression at will: selection and design of zinc finger domains recognizing each of the 5′-GNN-3′DNA target sequences. Proc Natl Acad Sci USA, 1999. 96(6): p. 2758-63.
48. Sewell, R. F., P. E. Brenchley, and N. P. Mallick, Human mononuclear cells contain an endoglycosidase specific for heparan sulphate glycosaminoglycan demonstrable with the use of a specific solid-phase metabolically radiolabelled substrate. Biochem J, 1989. 264(3): p. 777-83.
49. Shively, J. E. and H. E. Conrad, Formation of anhydrosugars in the chemical depolymerization of heparin. Biochemistry, 1976. 15(18): p. 3932-42.
50. Tempest, P. R., et al., Reshaping a human monoclonal antibody to inhibit human respiratory syncytial virus infection in vivo. Biotechnology (NY), 1991. 9(3): p. 266-71.
51. Toyoshima, M. et al. Human heparanase: Purification, characterization, cloning and expression. J. Biol. Chem. 274:29587-29590
52. Verhoeyen, M., C. Milstein, and G. Winter, Reshaping human antibodies: grafting an antilysozyme activity. Science, 1988. 239(4847): p. 1534-6
53. Verma, I. M., Gene therapy. Sci Am, 1990. 263(5): p. 68-72, 81-4.
54. Vettel, U., et al., Coordinate secretion and functional synergism of T cell-associated serine proteinase-1 (MTSP-1) and endoglycosidase(s) of activated T cells. Eur J Immunol, 1991. 21(9): p. 2247-51.
55. Van Regenmortel, M. H. V., 1986. Trends in Biological Sciences 11: 36-39.
56. Vlodavsky, I., et al., Expression of heparanase by platelets and circulating cells of the immune system: possible involvement in diapedesis and extravasation. Invasion Metastasis, 1992. 12(2): p. 112-27.
57. Vlodavsky I. et al., Mammalian heparanase: gene cloning, expression and function in tumor progression and metastasis., 1999 Nature Medicine. 5(7):793-802.
58. Wu, H., W. P. Yang, and C. F. Barbas, 3rd, Building zinc fingers by selection: toward a therapeutic application. Proc Natl Acad Sci U S A, 1995. 92(2): p. 344-8.
59. Yahalom, J., et al., Differentiating human leukemia cells express heparanase that degrades heparan sulfate in subendothelial extracellular matrix. Leuk Res, 1988. 12(9): p. 711-7.

### SUMMARY OF THE INVENTION

The present invention addresses the need identified above in that it provides isolated nucleic acid molecules encoding a heretofore unknown heparanase termed heparanase 11; constructs and recombinant host cells incorporating the isolated nucleic acid molecules; the heparanase II polypeptides encoded by the isolated nucleic acid molecules; antibodies to the heparanase II polypeptide; and methods of making and using all of the foregoing.

In one embodiment, the invention provides an isolated heparanase II polypeptide comprising the amino acid sequence set forth in SEQ ID NO: 2. It is understood that the polypeptide of SEQ ID NO:2 may be subject to specific proteolytic processing events resulting in a number of polypeptide species. Unless otherwise indicated, any reference herein to a “heparanase II polypeptide” will be understood to encompass pre-pro-heparanase II, pro-heparanase II, and both the 8 kDa and the 50 kDa subunits of the heparanase II enzyme and other species resulting from specific proteolytic processing events of heparanase II and functional equivalents including conservative amino acid substitutions. It is further understood that the human heparanase II enzyme may exist in a two-chain form with fragments resulting from specific proteolytic processing events remaining associated with each other.

Pre-pro-heparanase II refers to the amino acid sequence comprising SEQ ID NO:2, Pre-pro-heparanase includes a leader sequence, and can be processed further by proteolysis to remove the leader sequence and to remove internal amino acids yielding both the 8 kDa and the 50 kDa subunits of the human heparanase II enzyme;

Pro-heparanase II refers to the full-length molecule of SEQ ID NO:2 with the signal sequence removed (amino acids 1-41). Pro-heparanase therefore refers to a single chain polypeptide comprising the amino acid sequence at residues 42 through 534 of SEQ ID NO:2.

Pro-heparanase II can be further processed by proteolysis to remove 32 internal amino acids yielding both the 8 kDa and the 50 kDa subunits of the heparanase II enzyme. The 8 kDa subunit refer to a single chain polypeptide comprising the amino acid sequence at residues 42 through 129 of SEQ ID NO:2. The 50 kDa subunit refers to a single chain polypeptide comprising the amino acid sequence at residues 162 through 534 of SEQ ID NO:2.

In addition, it should be recognized that generally proteolytic processing as the result of two endoproteolytic cleavages produces the 8kDa and the 50 kDa subunits whereas a single proteolytic cut at either position results in two polypeptide chains of slightly different molecular weights. A proteolytic cut at the more amino terminal processing site results in two polypeptide chains one comprising the amino acid sequence at residues 130 through 534 of SEQ ID NO:2, the other comprising the amino acid sequence at residues 42 through 129 of SEQ ID NO:2.

In addition the invention provides a fragment comprising an epitope of the heparanse II polypeptide. By “epitope specific to” is meant a portion of the heparanase II enzyme that is recognizable by an antibody that is specific for heparanase II polypeptide, as defined in detail below. Another embodiment comprises an isolated polypeptide comprising the complete amino acid sequence set forth in SEQ ID NO: 2.

The cDNA sequence and predicted amino acid sequence of human heparanase II is reproduced below.

| | |
|---|---|
| CAGGTTTTAAATCAGAGGGATTGAATGAGGGTGCTTTGTGCCTTCCCTGAAGCCATGCCC | 60 |
| M R V L C A F P E A M P | 12 |
| TCCAGCAACTCCCGCCCCCCCGCGTGCCTAGCCCCGGGGGCTCTCTACTTGGCTCTGTTG | 120 |
| S S N S R P P A C L A P G A L Y L A L L | 32 |
| CTCCATCTCTCCCTTTCCTCCCAGGCTGGAGACAGGAGACCCTTGCCTGTAGACAGAGCT | 180 |
| L H L S L S S Q A G D R R P L P V D R A | 52 |
| GCAGGTTTGAAGGAAAAGACCCTGATTCTACTTGATGTGAGCACCAAGAACCCAGTCAGG | 240 |
| A G L K E K T L I L L D V S T K N P V R | 72 |
| ACAGTCAATGAGAACTTCCTCTCTCTGCAGCTGGATCCGTCCATCATTCATGATGGCTGG | 300 |
| T V N E N F L S L Q L D P S I I H D G W | 92 |
| CTCGATTTCCTAAGCTCCAAGCGCTTGGTGACCCTGGCCCGGGGACTTTCGCCCGCCTTT | 360 |
| L D F L S S K R L V T L A R G L S P A F | 112 |
| CTGCGCTTCGGGGGCAAAAGGACCGACTTCCTGCAGTTCCAGAACCTGAGGAACCCGGCG | 420 |
| L R F G G K R T D F L Q F Q N L R N P A | 132 |
| AAAAGCCGCGGGGGCCCGGGCCCGGATTACTATCTCAAAAACTATGAGGATGACATTGTT | 480 |
| K S R G G P G P D Y Y L K N Y E D D I V | 152 |
| CGAAGTGATGTTGCCTTAGATAAACAGAAAGGCTGCAAGATTGCCCAGCACCCTGATGTT | 540 |
| R S D V A L D K Q K G C K I A Q H P D V | 172 |
| ATGCTGGAGCTCCAAAGGGAGAAGGCAGCTCAGATGCATCTGGTTCTTCTAAAGGAGCAA | 600 |
| M L E L Q R E K A A Q M H L V L L K E Q | 192 |
| TTCTCCAATACTTACAGTAATCTCATATTAACAGAGCCAAATAACTATCGGACCATGCAT | 660 |
| F S N T Y S N L I L T E P N N Y R T M H | 212 |
| GGCCGGGCAGTAAATGGCAGCCAGTTGGGAAAGGATTACATCCAGCTGAAGAGCCTGTTG | 720 |
| G R A V N G S Q L G K D Y I Q L K S L L | 232 |
| CAGCCCATCCGGATTTATTCCAGAGCCAGCTTATATGGCCCTAATATTGGGCGGCCGAGG | 780 |
| Q P I R I Y S R A S L Y G P N I G R P R | 252 |
| AAGAATGTCATCGCCCTCCTAGATGGATTCATGAAGGTGGCAGGAAGTACAGTAGATGCA | 840 |
| K N V I A L L D G F M K V A G S T V D A | 272 |
| GTTACCTGGCAACATTGCTACATTGATGGCCGGGTGGTCAAGGTGATGGACTTCCTGAAA | 900 |
| V T W Q H C Y I D G R V V K V M D F L K | 292 |
| ACTCGCCTGTTAGACACACTCTCTGACCAGATTAGGAAAATTCAGAAAGTGGTTAATACA | 960 |
| T R L L D T L S D Q I R K I Q K V V N T | 312 |
| TACACTCCAGGAAAGAAGATTTGGCTTGAAGGTGTGGTGACCACCTCAGCTGGAGGCACA | 1020 |
| Y T P G K K I W L E G V V T T S A G G T | 332 |
| AACAATCTATCCGATTCCTATGCTGCAGGATTCTTATGGTTGAACACTTTAGGAATGCTG | 1080 |
| N N L S D S Y A A G F L W L N T L G M L | 352 |
| GCCAATCAGGGCATTGATGTCGTGATACGGCACTCATTTTTTGACCATGGATACAATCAC | 1140 |
| A N Q G I D V V I R H S F F D H G Y N H | 372 |
| CTCGTGGACCAGAATTTTAACCCATTACCAGACTACTGGCTCTCTCTCCTCTACAAGCGC | 1200 |
| L V D Q N F N P L P D Y W L S L L Y K R | 392 |
| CTGATCGGCCCCAAAGTCTTGGCTGTGCATGTGGCTGGGCTCCAGCGGAAGCCACGGCCT | 1260 |
| L I G P K V L A V H V A G L Q R K P R P | 412 |
| GGCCGAGTGATCCGGGACAAACTAAGGATTTATGCTCACTGCACAAACCACCACAACCAC | 1320 |
| G R V I R D K L R I Y A H C T N H H N H | 432 |
| AACTACGTTCGTGGGTCCATTACACTTTTTATCATCAACTTGCATCGATCAAGAAAGAAA | 1380 |
| N Y V R G S I T L F I I N L H R S R K K | 452 |
| ATCAAGCTGGCTGGGACTCTCAGAGACAAGCTGGTTCACCAGTACCTGCTGCAGCCCTAT | 1440 |
| I K L A G T L R D K L V H Q Y L L Q P Y | 472 |
| GGGCAGGAGGGCCTAAAGTCCAAGTCAGTGCAACTGAATGGCCAGCCCTTAGTGATGGTG | 1500 |
| G Q E G L K S K S V Q L N G Q P L V M V | 492 |
| GACGACGGGACCCTCCCAGAATTGAAGCCCCGCCCCCTTCGGGCCGGCCGGACATTGGTC | 1560 |
| D D G T L P E L K P R P L R A G R T L V | 512 |
| ATCCCTCCAGTCACCATGGGCTTTTTTGTGGTCAAGAATGTCAATGCTTTGGCCTGCCGC | 1620 |
| I P P V T M G F F V V K N V N A L A C R | 532 |
| TACCGATAAGCTATCCTCACACTCATGGCTACCAGTGGGCCTGCTGGGCTGCTTCCACTC | 1680 |
| Y R | 534 |
| CTCCACTCCAGTAGTATCCTCTGTTTTCAGACATCCTAGCAACCAGCCCCTGCTGCCCCA | 1740 |
| TCCTGCTGGAATCAACACAGACTTGCTCTCCAAAGAGACTAAATGTCATAGCGTGATCTT | 1800 |
| AGCCTAGGTAGGCCACATCCATCCCAAAGGAAAATGTAGACATCACCTGTACCTATATAA | 1860 |
| GGATAAAGGCATGTGTATAGAGCAGAATGTTTCTCTTCATGTGCACTATGAAAACGAGCT | 1920 |
| GACAGCACACTCCCAGGAGAAATGTTTCCAGACAACTCCCCATGATCCTGTCACACAGCA | 1980 |
| TTATAACCACAAATCCAAACCTTAGCCTGCTGCTGCTGCTGCCCTCAGAGGAAGATGAGG | 2040 |
| AAGGAAAAAAAACTGGGTGGACCTACAAAAACCCATCCTCTCCCAACTCCTTCTTCTCTG | 2100 |
| CCTCTTTCTTGCTGCTGCCCTGAGTTTTTTGACACATCTCTTTCCATAGGGGAGTAATGG | 2160 |
| GTGTGTCAGCCCTGGCCTGCTGGGAGAGCTGTTTATATGATTTCCCGGCTGATGTATGAG | 2220 |
| CGTGCGCACCTGGGTTCCTCACAGTGGCATCCATCACTGGCAGTTCTTCTGGGAAGCGGG | 2280 |
| TGCTTCAAAAGTAAAATTACAATCACACTCCAAAAAAAAAAAAAAA | 2326 |

Although SEQ ID NOS: 1 and 2 provides particular human polynucleotide and polypeptide sequences, the invention is intended to include within its scope other human allelic variants; non-human mammalian forms of heparanase II, and other vertebrate forms of heparanase 11.

In another embodiment, the invention provides isolated polynucleotides (e.g., cDNA, genomic DNA, synthetic DNA, RNA, or combinations thereof, single or double stranded) that comprise a nucleotide sequence encoding the amino acid sequence of the polypeptides of the invention. Such polynucleotides are useful for recombinantly expressing the enzyme and also for detecting expression of the enzyme in cells (e.g., using Northern hybridization and in situ hybridization assays). Such polynucleotides also are useful to design antisense and other molecules for the suppression of the expression of heparanase II in a cultured cell or tissue or in an animal, for therapeutic purposes or to provide a model for diseases characterized by aberrant heparanase II expression. Specifically excluded from the definition of polynucleotides of the invention are entire isolated chromosomes from native host cells from which the polynucleotide was originally derived. The polynucleotide set forth in SEQ ID NO: 1 corresponds to naturally occurring heparanase II sequence. It will be appreciated that numerous other sequences exist that also encode heparanase II of SEQ ID NO: 2 due to the well-known degeneracy of the universal genetic code. In another embodiment, the invention is directed to all of the degenerate heparanase II-encoding sequences other than the sequence set forth in SEQ ID NO: 1.

The invention also provides an isolated polynucleotide comprising a nucleotide sequence that encodes a mammalian heparanase II enzyme, wherein the polynucleotide hybridizes to the nucleotide sequence set forth in SEQ ID NO: 1 or the non-coding strand complementary thereto, under the following hybridization conditions:
(a) hybridization for 16 hours at 42° C. in a hybridization solution comprising 50% formamide, 1% SDS, IM NaCI, 10% Dextran sulfate; and
(b) washing 2 times for 30 minutes at 60° C. in a wash solution comprising 0.1% SSC, 1% SDS.

One polynucleotide of the invention comprises the sequence set forth in SEQ ID NO: 1, which comprises a human heparanase II encoding DNA sequence:

In a related embodiment, the invention provides vectors comprising a polynucleotide of the invention. Such vectors are useful, e.g., for amplifying the polynucleotides in host cells to create useful quantities thereof. In other embodiments, the vector is an expression vector wherein the polynucleotide of the invention is operatively linked to a polynucleotide comprising an expression control sequence. Such vectors are useful for recombinant production of polypeptides of the invention.

In another related embodiment, the invention provides host cells that are transformed or transfected (stably or transiently) with polynucleotides of the invention or vectors of the invention. As stated above, such host cells are useful for amplifying the polynucleotides and also for expressing the heparanase II enzyme polypeptide or fragment thereof encoded by the polynucleotide.

In still another related embodiment, the invention provides a method for producing a heparanase II polypeptide (or fragment thereof) comprising the steps of growing a host cell of the invention in a nutrient medium and isolating the polypeptide or variant thereof from the cell or the medium.

In still another embodiment, the invention provides an antibody that is specific for the heparanase II enzyme of the invention. Antibody specificity is described in greater detail below. However, it should be emphasized that antibodies that can be generated from polypeptides that have previously been described in the literature and that are capable of fortuitously cross-reacting with heparanase II (e.g., due to the fortuitous existence of a similar epitope in both polypeptides) are considered “cross-reactive” antibodies. Such cross-reactive antibodies are not antibodies that are “specific” for heparanase II. The determination of whether an antibody is specific for heparanase II or is cross-reactive with another known enzyme is made using Western blotting assays or several other assays well known in the literature. For identifying cells that express heparanase II and also for modulating heparanase II activity, antibodies that specifically bind to the active site of heparanase II are particularly useful but of course, antibodies binding other epitopes are contemplated as part of the invention as well.

In one variation, the invention provides monoclonal antibodies. Hybridomas that produce such antibodies also are intended as aspects of the invention. In yet another variation, the invention provides a humanized antibody. Humanized antibodies are useful for in vivo therapeutic indications.

In another variation, the invention provides a cell-free composition comprising polyclonal antibodies, wherein at least one of the antibodies is an antibody of the invention specific for heparanase II. Antisera isolated from an animal is an exemplary composition, as is a composition comprising an antibody fraction of an antisera that has been resuspended in water or in another diluent, excipient, or carrier.

In still another related embodiment, the invention provides an anti-idiotypic antibody specific for an antibody that is specific for heparanase II.

It is well known that antibodies contain relatively small antigen binding domains that can be isolated chemically or by recombinant techniques. Such domains are useful heparanase II binding molecules themselves, and also may be reintroduced into human antibodies, or fused to toxins or other polypeptides. Thus, in still another embodiment, the invention provides a polypeptide comprising a fragment of a heparanase II-specific antibody, wherein the fragment and the polypeptide bind to the heparanase II active site. By way of non-limiting example, the invention provides polypeptides that are single chain antibodies and CDR-grafted antibodies.

Also within the scope of the invention are compositions comprising polypeptides, polynucleotides, or antibodies of the invention that have been formulated with, e.g., a pharmaceutically acceptable carrier.

The invention also provides methods of using antibodies of the invention. For example, the invention provides a method for inhibiting the enzymatic activity of a heparanase II enzyme comprising the step of contacting enzyme with an antibody specific for the enzyme's active site, under conditions wherein the antibody binds the enzyme and inhibits its activity.

The invention also provides assays to identify compounds that alter heparanase II enzymatic activity. One such assay comprises the steps of: (a) contacting a composition comprising heparanase II enzyme with a compound suspected of altering heparanase II activity; and (b) measuring the enzymatic activity of the heparanase II in the presence and the absence of the compound suspected of altering the enzymatic activity of heparanase II and (c) comparing the measured enzymatic activity in the presence and the absence of the compound, whereby a change in heparanase activity indicates that the compound has altered the activity of said heparanase activity. In one variation, the composition comprises a cell expressing heparanase II. In another variation, isolated heparanase is employed.

The invention also provides a method for treating a disease state comprising the step of administering to a mammal in need of such treatment an amount of an agent sufficient to alter heparanase II enzymatic activity in the tissues of said mammal In addition to the foregoing, the invention includes, as an additional aspect, all embodiments of the invention narrower in scope in any way than the variations specifically mentioned above. Although the applicant(s) invented the full scope of the claims appended hereto, the claims appended hereto are not intended to encompass within their scope the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

### BRIEF DESCRIPTION OF THE FIGURES

FIG. 1 Predicted amino acid sequence of human heparanase II depicting functional motifs. The signal peptide is shown in bold, canonical acceptor sites for N-linked glycosylation are italicized in bold and double underlined, and predicted sites for phosphorylation by protein kinase C are shown in bold and underlined.

FIG. 2 Clustal W multiple sequence alignment of human heparanase I and human heparanase II

FIG. 3—Northern Blot showing the tissue distribution of human heparanase II

### BRIEF DESCRIPTION OF THE SEQUENCE LISTINGS

SEQ ID NO: 1—cDNA sequence encoding human preproheparanase II

SEQ ID NO: 2—predicted amino acid sequence of preprohepaparanase 11

SEQ ID NO: 3—PCR primer, Example 1

SEQ ID NO: 4—PCR primer, Example 1

SEQ ID NO: 5—PCR primer, Example 1

### DETAILED DESCRIPTION OF THE INVENTION

The present invention provides isolated polynucleotides (e.g., DNA sequences and RNA transcripts, both sense and complementary antisense strands, both single and double-stranded, including splice variants thereof) encoding an enzyme referred to herein as heparanase II. DNA polynucleotides of the invention include genomic DNA, cDNA, and DNA that has been chemically synthesized in whole or in part. “Synthesized” as used herein and understood in the art, refers to polynucleotides produced by purely chemical, as opposed to enzymatic, methods. “Wholly” synthesized DNA sequences are therefore produced entirely by chemical means, and “partially” synthesized DNAs embrace those wherein only portions of the resulting DNA were produced by chemical means. “Isolated” as used herein and as understood in the art, whether referring to “isolated” polynucleotides or polypeptides, is taken to mean separated from the original cellular environment in which the polypeptide or nucleic acid is normally found. As used herein therefore, by way of example only, a transgenic animal or a recombinant cell line constructed with a polynucleotide of the invention, makes use of the “isolated” nucleic acid.

Genomic DNA of the invention comprises the protein coding region for a polypeptide of the invention and is also intended to include allelic variants thereof. It is widely understood that, for many genes, genomic DNA is transcribed into RNA transcripts that undergo one or more splicing events wherein intron (i.e., non-coding regions) of the transcripts are removed, or “spliced out.” RNA transcripts that can be spliced by alternative mechanisms, and therefore be subject to removal of different RNA sequences but still encode a heparanase II polypeptide, are referred to in the art as splice variants which are embraced by the invention. Splice variants comprehended by the invention therefore are encoded by the same original genomic DNA sequences but arise from distinct mRNA transcripts. Allelic variants are modified forms of a wild type gene sequence, the modification resulting from recombination during chromosomal segregation or exposure to conditions which give rise to genetic mutation. Allelic variants, like wild type genes, are naturally occurring sequences (as opposed to non-naturally occurring variants which arise from in vitro manipulation).

The invention also comprehends cDNA that is obtained through reverse transcription of an RNA polynucleotide encoding heparanase II (conventionally followed by second strand synthesis of a complementary strand to provide a double-stranded DNA).

A DNA sequence encoding a human heparanase II polypeptide is set out in SEQ ID NO: 1. The worker of skill in the art will readily appreciate that the DNA of the invention comprises a double stranded molecule, for example the molecule having the sequence set forth in SEQ ID NO: 1 along with the complementary molecule (the “non-coding strand” or “complement”) having a sequence deducible from the sequence of SEQ ID NO: 1 according to Watson-Crick base pairing rules for DNA. Also contemplated by the invention are other polynucleotides encoding the heparanase II polypeptide of SEQ ID NO: 2, which differ in sequence from the polynucleotide of SEQ ID NO: 1 by virtue of the well-known degeneracy of the universal genetic code.

The invention further embraces species, preferably mammalian, homologs of the human heparanase II DNA. Species homologs, sometimes referred to as “orthologs,” in general, share at least 35%, at least 40%, at least 45%, at least 50%, at least 60%, at least 65%, at least 70%, at least 75%, at least 80%, at least 85%, at least 90%, at least 95%, at least 98%, or at least 99% homology with SEQ ID NO: 1 of the invention. Percent sequence “homology” with respect to polynucleotides of the invention is defined herein as the percentage of nucleotide bases in the candidate sequence that are identical to nucleotides in the heparanase II sequence set forth in SEQ ID NO: 1, after aligning the sequences and introducing gaps, if necessary, to achieve the maximum percent sequence identity. The percentage of sequence between a native and a variant human heparanase sequence may also be determined, for example, by comparing the two sequences using any of the computer programs commonly employed for this purpose, such as the Gap program (Wisconsin Sequence Analysis Package, Version 8 for Unix, Genetics Computer Group, University Research Park, Madison Wis.), which uses the algorithm of Smith and Waterman (Adv. Appl. Math. 2: 482-489 (1981)).

The polynucleotide sequence information provided by the invention makes possible large scale expression of the encoded polypeptide by techniques well known and routinely practiced in the art. Polynucleotides of the invention also permit identification and isolation of polynucleotides encoding related heparanase II polypeptides, such as human allelic variants and species homologs, by well known techniques including Southern and/or Northern hybridization, and polymerase chain reaction (PCR). Examples of related polynucleotides include human and non-human genomic sequences, including allelic variants, as well as polynucleotides encoding polypeptides homologous to heparanase II and structurally related polypeptides sharing one or more biological, immunological, and/or physical properties of heparanase II. Non-human species genes encoding proteins homologous to heparanase II can also be identified by Southern and/or PCR analysis and are useful in animal models for heparanase II disorders. Knowledge of the sequence of a human heparanase II DNA also makes possible through use of Southern hybridization or polymerase chain reaction (PCR) the identification of genomic DNA sequences encoding heparanase II expression control regulatory sequences such as promoters, operators, enhancers, repressors, and the like. Polynucleotides of the invention are also useful in hybridization assays to detect the capacity of cells to express heparanase II. Polynucleotides of the invention may also be the basis for diagnostic methods useful for identifying a genetic alteration(s) in a heparanase II locus that underlies a disease state or states, which information is useful both for diagnosis and for selection of therapeutic strategies.

The disclosure herein of a full length polynucleotide encoding a heparanase II polypeptide makes readily available to the worker of ordinary skill in the art every possible fragment of the full length polynucleotide. The invention therefore provides fragments of heparanase II -encoding polynucleotides comprising at least 14-15, and preferably at least 18, 20, 25, 50, or 75 consecutive nucleotides of a polynucleotide encoding heparanase II. Preferably, fragment polynucleotides of the invention comprise sequences unique to the heparanase II-encoding polynucleotide sequence, and therefore hybridize under highly stringent or moderately stringent conditions only (i.e., “specifically”) to polynucleotides encoding heparanase II (or fragments thereof). Polynucleotide fragments of genomic sequences of the invention comprise not only sequences unique to the coding region, but also include fragments of the full length sequence derived from introns, regulatory regions, and/or other non-translated sequences. Sequences unique to polynucleotides of the invention are recognizable through sequence comparison to other known polynucleotides, and can be identified through use of alignment programs routinely utilized in the art, e.g., those made available in public sequence databases. Such sequences also are recognizable from Southern hybridization analyses to determine the number of fragments of genomic DNA to which a polynucleotide will hybridize. Polynucleotides of the invention can be labeled in a manner that permits their detection, including radioactive, fluorescent, and enzymatic labeling.

Fragment polynucleotides are particularly useful as probes for detection of full length or other fragment heparanase II polynucleotides. One or more fragment polynucleotides can be included in kits that are used to detect the presence of a polynucleotide encoding heparanase II, or used to detect variations in a polynucleotide sequence encoding heparanase II.

The invention also embraces DNAs encoding heparanase II polypeptides which DNAs hybridize under moderately stringent or high stringency conditions to the non-coding strand, or complement, of the polynucleotide in SEQ ID NO: 1.

Exemplary highly stringent hybridization conditions are as follows: hybridization at 42° C. in a hybridization solution comprising 50% formamide, 1% SDS, 1M NaCl, 10% Dextran sulfate, and washing twice for 30 minutes at 60° C. in a wash solution comprising 0.1×SSC and 1% SDS. It is understood in the art that conditions of equivalent stringency can be achieved through variation of temperature and buffer, or salt concentration as described Ausubel, et al. (Eds.), Protocols in Molecular Biology, John Wiley & Sons (1994), pp. 6.0.3 to 6.4.10. Modifications in hybridization conditions can be empirically determined or precisely calculated based on the length and the percentage of guanosine/cytosine (GC) base pairing of the probe. The hybridization conditions can be calculated as described in Sambrook, et al., (Eds.), Molecular Cloning: A Laboratory Manual, Cold Spring Harbor Laboratory Press: Cold Spring Harbor, N.Y. (1989), pp. 9.47 to 9.51.

Autonomously replicating recombinant expression constructs such as plasmid and viral DNA vectors incorporating polynucleotides of the invention are also provided. Expression constructs wherein heparanase II -encoding polynucleotides are operatively linked to an endogenous or exogenous expression control DNA sequence and a transcription terminator are also provided. Expression control DNA sequences include promoters, enhancers, and operators, and are generally selected based on the expression systems in which the expression construct is to be utilized. Promoter and enhancer sequences are generally selected for the ability to increase gene expression, while operator sequences are generally selected for the ability to regulate gene expression. Expression constructs of the invention may also include sequences encoding one or more selectable markers that permit identification of host cells bearing the construct. Expression constructs may also include sequences that facilitate, and preferably promote, homologous recombination in a host cell. Constructs of the invention also include sequences necessary for replication in a host cell.

Expression constructs are preferably utilized for production of an encoded protein, but also may be utilized simply to amplify a heparanase II -encoding polynucleotide sequence.

According to another aspect of the invention, host cells are provided, including prokaryotic and eukaryotic cells, comprising a polynucleotide of the invention (or vector of the invention) in a manner which permits expression of the encoded heparanase II polypeptide. Polynucleotides of the invention may be introduced into the host cell as part of a circular plasmid, or as linear DNA comprising an isolated protein coding region or a viral vector. Methods for introducing DNA into the host cell well known and routinely practiced in the art include transformation, transfection, electroporation, nuclear injection, or fusion with carriers such as liposomes, micelles, ghost cells, and protoplasts. Expression systems of the invention include bacterial, yeast, fungal, plant, insect, invertebrate, and mammalian cells systems.

Suitable host cells for expression of human heparanase polypeptides include prokaryotes, yeast, and higher eukaryotic cells. Suitable prokaryotic hosts to be used for the expression of human heparanase include but are not limited to bacteria of the genera Escherichia, Bacillus, and Salmonella, as well as members of the genera Pseudomonas, Streptomyces, and Staphylococcus.

The isolated nucleic acid molecules of the invention are preferably cloned into a vector designed for expression in eukaryotic cells, rather than into a vector designed for expression in prokaryotic cells. Eukaryotic cells are sometimes preferred for expression of genes obtained from higher eukaryotes because the signals for synthesis, processing, and secretion of these proteins are usually recognized, whereas this is often not true for prokaryotic hosts (Ausubel, et al., ed., in Short Protocols in Molecular Biology, 2nd edition, John Wiley & Sons, publishers, pg.16-49, 1992.). In the case of the human heparanase II, there are 2 consensus sequences for N-linked glycosylation, and other sites of post-translational modification can be predicted for protein kinase C phosphorylation and O-glycosylation. Eukaryotic hosts may include, but are not limited to, the following: insect cells, African green monkey kidney cells (COS cells), Chinese hamster ovary cells (CHO cells), human 293 cells, and murine 3T3 fibroblasts.

Expression vectors for use in prokaryotic hosts generally comprise one or more phenotypic selectable marker genes. Such genes generally encode, e.g., a protein that confers antibiotic resistance or that supplies an auxotrophic requirement. A wide variety of such vectors are readily available from commercial sources. Examples include pSPORT vectors, pGEM vectors (Promega), pPROEX vectors (LTI, Bethesda, Md.), Bluescript vectors (Stratagene), and pQE vectors (Qiagen).

Human heparanase may also be expressed in yeast host cells from genera including Saccharomyces, Pichia, and Kluveromyces. Yeast hosts include S. cerevisiae and P. pastoris. Yeast vectors will often contain an origin of replication sequence from a 2 micron yeast plasmid, an autonomously replicating sequence (ARS), a promoter region, sequences for polyadenylation, sequences for transcription termination, and a selectable marker gene. Vectors replicable in both yeast and E. coli (termed shuttle vectors) may also be used. In addition to the above-mentioned features of yeast vectors, a shuttle vector will also include sequences for replication and selection in E. coli. Direct secretion of human heparanase II polypeptides expressed in yeast hosts may be accomplished by the inclusion of nucleotide sequence encoding the yeast factor leader sequence at the 5′ end of the human heparanase II-encoding nucleotide sequence.

Insect host cell culture systems may also be used for the expression of human heparanase II polypeptides. In another embodiment, the human heparanase II polypeptides of the invention are expressed using a baculovirus expression system. Further information regarding the use of baculovirus systems for the expression of heterologous proteins in insect cells are reviewed by Luckow and Summers, Bio/Technology 6:47 (1988).

In another embodiment, the heparanase II polypeptide is expressed in mammalian host cells. Non-limiting examples of suitable mammalian cell lines include the COS-7 line of monkey kidney cells (Gluzman et al., Cell 23:175 (1981)), Chinese hamster ovary (CHO) cells, and human 293 cells.

The choice of a suitable expression vector for expression of the heparanase II polypeptides of the invention will of course depend upon the specific host cell to be used, and is within the skill of the ordinary artisan. Examples of suitable expression vectors include pcDNA3 (Invitrogen) and pSVL (Pharmacia Biotech). Expression vectors for use in mammalian host cells may include transcriptional and translational control sequences derived from viral genomes. Commonly used promoter sequences and enhancer sequences which may be used in the present invention include, but are not limited to, those derived from human cytomegalovirus (CMV), Adenovirus 2, Polyoma virus, and Simian virus 40 (SV40). Methods for the construction of mammalian expression vectors are disclosed, for example, in Okayama and Berg (Mol. Cell. Biol. 3:280 (1983)); Cosman et al. (Mol. Immunol. 23:935 (1986)); Cosman et al. (Nature 312:768 (1984)); EP-A-0367566; and WO 91/18982.

Host cells of the invention are a valuable source of immunogen for development of antibodies specifically immunoreactive with heparanase II. Host cells of the invention are also useful in methods for large scale production of heparanase II polypeptides wherein the cells are grown in a suitable culture medium and the desired polypeptide products are isolated from the cells or from the medium in which the cells are grown by purification methods known in the art, e.g., conventional chromatographic methods including immunoaffinity chromatography, enzyme affinity chromatography, hydrophobic interaction chromatography, lectin affinity chromatography, size exclusion filtration, cation or anion exchange chromatography, high pressure liquid chromatography (HPLC), reverse phase HPLC, and the like. Still other methods of purification include those wherein the desired protein is expressed and isolated as a fusion protein having a specific tag, label, or chelating moiety that is recognized by a specific binding partner or agent. The isolated protein can be cleaved to yield the desired protein, or be left as an intact fusion protein. Cleavage of the fusion component may produce a form of the desired protein having additional amino acid residues as a result of the cleavage process.

Knowledge of heparanase II DNA sequences allows for modification of cells to permit, or increase, expression of endogenous heparanase II. Cells can be modified (e.g., by homologous recombination) to provide increased expression by replacing, in whole or in part, the naturally occurring heparanase II promoter with all or part of a heterologous promoter so that the cells express heparanase II at higher levels. The heterologous promoter is inserted in such a manner that it is operatively linked to endogenous heparanase II encoding sequences. [See, for example, PCT International Publication No. WO 94/12650, PCT International Publication No. WO 92/20808, and PCT International Publication No. WO 91/09955.] It is also contemplated that, in addition to heterologous promoter DNA, amplifiable marker DNA (e.g., ada, dhfr, and the multifunctional CAD gene which encodes carbamyl phosphate synthase, aspartate transcarbamylase, and dihydroorotase) and/or intron DNA may be inserted along with the heterologous promoter DNA. If linked to the heparanase II coding sequence, amplification of the marker DNA by standard selection methods results in co-amplification of the heparanase II coding sequences in the cells.

The DNA sequence information provided by the present invention also makes possible the development through, e.g. homologous recombination or “knock-out” strategies [Capecchi, Science 244:1288-1292 (1989)], of animals that fail to express functional heparanase II or that express a variant of heparanase II. Such animals (especially small laboratory animals such as rats, rabbits, and mice) are useful as models for studying the in vivo activities of heparanase II and modulators of heparanase II.

Also made available by the invention are anti-sense polynucleotides which recognize and hybridize to polynucleotides encoding heparanase II. Full length and fragment anti-sense polynucleotides are provided. Fragment anti-sense molecules of the invention include (i) those which specifically recognize and hybridize to heparanase II (as determined by sequence comparison of DNA encoding heparanase II to DNA encoding other known molecules). Identification of sequences unique to heparanase II -encoding polynucleotides, can be deduced through use of any publicly available sequence database, and/or through use of commercially available sequence comparison programs. The uniqueness of selected sequences in an entire genome can be further verified by hybridization analyses. After identification of the desired sequences, isolation through restriction digestion or amplification using any of the various polymerase chain reaction techniques well known in the art can be performed. Anti-sense polynucleotides are particularly relevant to regulating expression of heparanase II by those cells expressing heparanase II mRNA.

Antisense nucleic acids (preferably 10 to 20 base pair oligonucleotides) capable of specifically binding to heparanase II expression control sequences or heparanase II RNA are introduced into cells (e.g., by a viral vector or colloidal dispersion system such as a liposome). The antisense nucleic acid binds to the heparanase II target nucleotide sequence in the cell and prevents transcription or translation of the target sequence. Phosphorothioate and methylphosphonate antisense oligonucleotides are specifically contemplated for therapeutic use by the invention. The antisense oligonucleotides may be further modified by poly-L-lysine, transferrin polylysine, or cholesterol moieties at their 5′ end. Suppression of heparanase II expression at either the transcriptional or translational level is useful to generate cellular or animal models for diseases characterized by aberrant heparanase II expression or as a therapeutic modality.

The heparanase II sequences taught in the present invention facilitate the design of novel transcription factors for modulating heparanase II expression in native cells and animals, and cells transformed or transfected with heparanase II polynucleotides. For example, the Cys2-His2 zinc finger proteins, which bind DNA via their zinc finger domains, have been shown to be amenable to structural changes that lead to the recognition of different target sequences. These artificial zinc finger proteins recognize specific target sites with high affinity and low dissociation constants, and are able to act as gene switches to modulate gene expression. Knowledge of the particular heparanase II target sequence of the present invention facilitates the engineering of zinc finger proteins specific for the target sequence using known methods such as a combination of structure-based modeling and screening of phage display libraries [Segal et al., (1999) Proc Natl Acad Sci USA 96:2758-2763; Liu et al., (1997) Proc Natl Acad Sci USA 94:5525-30; Greisman and Pabo (1997) Science 275:657-61; Choo et al., (1997) J Mol Biol 273:525-32]. Each zinc finger domain usually recognizes three or more base pairs. Since a recognition sequence of 18 base pairs is generally sufficient in length to render it unique in any known genome, a zinc finger protein consisting of 6 tandem repeats of zinc fingers would be expected to ensure specificity for a particular sequence [Segal et al., (1999) Proc Natl Acad Sci USA 96:2758-2763]. The artificial zinc finger repeats, designed based on heparanase II sequences, are fused to activation or repression domains to promote or suppress heparanase II expression [Liu et al., (1997) Proc Natl Acad Sci USA 94:5525-30]. Alternatively, the zinc finger domains can be fused to the TATA box-binding factor (TBP) with varying lengths of linker region between the zinc finger peptide and the TBP to create either transcriptional activators or repressors [Kim et al., (1997) Proc Natl Acad Sci USA 94:3616-3620]. Such proteins, and polynucleotides that encode them, have utility for modulating heparanase II expression in vivo in both native cells, animals and humans; and/or cells transfected with heparanase II-encoding sequences. The novel transcription factor can be delivered to the target cells by transfecting constructs that express the transcription factor (gene therapy), or by introducing the protein. Engineered zinc finger proteins can also be designed to bind RNA sequences for use in therapeutics as alternatives to antisense or catalytic RNA methods [McColl et al., (1999) Proc Natl Acad Sci USA 96:9521-6; Wu et al., (1995) Proc Natl Acad Sci USA 92:344-348]. The present invention contemplates methods of designing such transcription factors based on the gene sequence of the invention, as well as customized zinc finger proteins, that are useful to modulate heparanase II expression in cells (native or transformed) whose genetic complement includes these sequences.

The invention also provides isolated mammalian heparanase II polypeptides encoded by a polynucleotide of the invention. The human heparanase II polypeptide amino acid sequence is set out in SEQ ID NO: 2.

The invention also embraces polypeptides that have at least 99%,at least 95%, at least 90%, at least 85%, at least 80%, at least 75%, at least 70%, at least 65%, at least 60%, at least 55% or at least 50% identity and/or homology to the polypeptide polypeptide set out in SEQ ID NO: 2. Percent amino acid sequence “identity” with respect to the polypeptide of SEQ ID NO: 2 is defined herein as the percentage of amino acid residues in the candidate sequence that are identical with the residues in the heparanase II sequence after aligning both sequences and introducing gaps, if necessary, to achieve the maximum percent sequence identity, and not considering any conservative substitutions as part of the sequence identity. Percent sequence “homology” with respect to the polypeptide of SEQ ID NO: 2 is defined herein as the percentage of amino acid residues in the candidate sequence that are identical with the residues in the heparanase II sequence after aligning the sequences and introducing gaps, if necessary, to achieve the maximum percent sequence identity, and also considering any conservative substitutions as part of the sequence identity.

In one aspect, percent homology is calculated as the percentage of amino acid residues in the smaller of two sequences which align with identical amino acid residue in the sequence being compared, when four gaps in a length of 100 amino acids may be introduced to maximize alignment (Dayhoff, in Atlas of Protein Sequence and Structure, Vol. 5, p. 124, National Biochemical Research Foundation, Washington, D.C. (1972), incorporated herein by reference].

Polypeptides of the invention may be isolated from natural cell sources or may be chemically synthesized, but are preferably produced by recombinant procedures involving host cells of the invention. Use of mammalian host cells is expected to provide for such post-translational modifications (e.g., glycosylation, truncation, lipidation, and phosphorylation) as may be needed to confer optimal biological activity on recombinant expression products of the invention. Glycosylated and non-glycosylated form of heparanase II polypeptides are embraced.

The invention also embraces variant (or analog) heparanase II polypeptides. In one example, insertion variants are provided wherein one or more amino acid residues supplement a heparanase II amino acid sequence. Insertions may be located at either or both termini of the protein, or may be positioned within internal regions of the heparanase II amino acid sequence. Insertional variants with additional residues at either or both termini can include for example, fusion proteins and proteins including amino acid tags or labels. Insertion variants include heparanase II polypeptides wherein one or more amino acid residues are added to a heparanase II acid sequence, or to a biologically active fragment thereof.

Variant products of the invention also include mature heparanase II products, i.e., heparanase II products wherein leader or signal sequences are removed, with additional amino terminal residues. The additional amino terminal residues may be derived from another protein, or may include one or more residues that are not identifiable as being derived from a specific proteins. heparanase II products with an additional methionine residue at position -1 (Met-1-heparanase II) are contemplated, as are variants with additional methionine and lysine residues at positions −2 and −1 (Met-2-Lys-1-heparanase II). Variants of heparanase II with additional Met, Met-Lys, Lys residues (or one or more basic residues in general) are particularly useful for enhanced recombinant protein production in bacterial host cell.

The invention also embraces heparanase II variants having additional amino acid residues which result from use of specific expression systems. For example, use of commercially available vectors that express a desired polypeptide as part of glutathione-S-transferase (GST) fusion product provides the desired polypeptide having an additional glycine residue at position −1 after cleavage of the GST component from the desired polypeptide. Another exemplary tag of this type is a poly-histidine sequence, generally around six histidine residues, that permits isolation of a compound so labeled using nickel chelation. Other labels and tags, such as the FLAG® tag (Eastman Kodak, Rochester, N.Y.), well known and routinely used in the art, are embraced by the invention. Variants which result from expression in other vector systems are also contemplated.

Insertional variants also include fusion proteins wherein the amino and/or carboxy termini of heparanase II is fused to another polypeptide.

In another aspect, the invention provides deletion variants wherein one or more amino acid residues in a heparanase II polypeptide are removed. Deletions can be effected at one or both termini of the heparanase II polypeptide, or with removal of one or more residues within the heparanase II amino acid sequence. Deletion variants, therefore, include all fragments of a heparanase II polypeptide.

The invention also embraces polypeptide fragments of the sequence set out in SEQ ID NO: 2 wherein the fragments maintain biological (e.g., ligand binding and/or intracellular signaling) immunological properties of a heparanase II polypeptide. Fragments comprising at least 5, 10, 15, 20, 25, 30, 35, or 40 consecutive amino acids of SEQ ID NO: 2 are comprehended by the invention. It is contemplated that polypeptide fragments can display antigenic properties unique to or specific for human heparanase II and its allelic and species homologs. Fragments of the invention having the desired biological and immunological properties can be prepared by any of the methods well known and routinely practiced in the art.

In still another aspect, the invention provides substitution variants of heparanase II polypeptides. Substitution variants include those polypeptides wherein one or more amino acid residues of a heparanase II polypeptide are removed and replaced with alternative residues. In one aspect, the substitutions are conservative in nature, however, the invention embraces substitutions that are also non-conservative. Conservative substitutions for this purpose may be defined as set out in Tables A, B, or C below.

Variant polypeptides include those wherein conservative substitutions have been introduced by modification of polynucleotides encoding polypeptides of the invention. Amino acids can be classified according to physical properties and contribution to secondary and tertiary protein structure. A conservative substitution is recognized in the art as a substitution of one amino acid for another amino acid that has similar properties. Exemplary conservative substitutions are set out in Table A (from WO 97/09433, page 10, published Mar. 13, 1997 (PCT/GB96/02197, filed Sep. 6, 1996), immediately below.

TABLE A
Conservative Substitutions I

| SIDE CHAIN | |
|---|---|
| CHARACTERISTIC | AMINO ACID |
| Aliphatic | |
| Non-polar | G A P |
| | I L V |
| Polar - uncharged | C S T M |
| | N Q |
| Polar - charged | D E |
| | K R |
| Aromatic | H F W Y |
| Other | N Q D E |

Alternatively, conservative amino acids can be grouped as described in Lehninger, [Biochemistry, Second Edition; Worth Publisers, Inc. NY:N.Y. (1975), pp. 71-77] as set out in table B, immediately below

TABLE B
Conservative Substitutions II

| SIDE CHAIN | |
|---|---|
| CHARACTERISTIC | AMINO ACID |
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

As still an another alternative, exemplary conservative substitutions are set out in Table C, immediately below.

TABLE C
Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met (M) | Leu, Phe, Ile |
| Phe (F) | Leu, Val, Ile, Ala |
| Pro (P) | Gly |
| Ser (S) | Thr |
| Thr (T) | Ser |
| Trp (W) | Tyr |
| Tyr (Y) | Trp, Phe, Thr, Ser |
| Val (V) | Ile, Leu, Met, Phe, Ala |

Variants that display enzymatic properties of native heparanase II and are expressed at higher levels and variants that provide for constitutive active enzyme are particularly useful in assays of the invention and also useful in cellular and animal models for diseases characterized by aberrant heparanase II expression activity.

It should be understood that the definition of polypeptides of the invention is intended to include polypeptides bearing modifications other than insertion, deletion, or substitution of amino acid residues. By way of example, the modifications may be covalent in nature, and include for example, chemical bonding with polymers, lipids, other organic, and inorganic moieties. Such derivatives may be prepared to increase circulating half-life of a polypeptide, or may be designed to improve targeting capacity for the polypeptide to desired cells, tissues, or organs.

Similarly, the invention further embraces heparanase II polypeptides that have been covalently modified to include one or more water soluble polymer attachments such as polyethylene glycol, polyoxyethylene glycol, or polypropylene glycol.

In a related embodiment, the present invention provides compositions comprising isolated polypeptides of the invention. Alternative compositions comprise, in addition to the polypeptide of the invention, a pharmaceutically acceptable (i.e., sterile and non-toxic) liquid, semisolid, or solid diluents that serve as pharmaceutical vehicles, excipients, or media. Any diluent known in the art may be used. Exemplary diluents include, but are not limited to, water, saline solutions, polyoxyethylene sorbitan monolaurate, magnesium stearate, methyl- and propylhydroxybenzoate, talc, alginates, starches, lactose, sucrose, dextrose, sorbitol, mannitol, glycerol, calcium phosphate, mineral oil, and cocoa butter.

Also comprehended by the present invention are antibodies (e.g., monoclonal and polyclonal antibodies, single chain antibodies, chimeric antibodies, bifunctional/bispecific antibodies, humanized antibodies, human antibodies, and complementary determining region (CDR)-grafted antibodies, including compounds which include CDR sequences which specifically recognize a polypeptide of the invention) specific for heparanase II or fragments thereof. Antibodies of the invention include human antibodies which are produced and identified according to methods described in WO93/11236, published Jun. 20, 1993, which is incorporated herein by reference in its entirety. Antibody fragments, including Fab, Fab′, F(ab′)2, and Fv, are also provided by the invention. The term “specific for,” when used to describe antibodies of the invention, indicates that the variable regions of the antibodies of the invention recognize and bind heparanase II polypeptides exclusively (i.e., able to distinguish heparanase II polypeptides from other known polypeptides by virtue of measurable differences in binding affinity, despite the possible existence of localized sequence identity, homology, or similarity between heparanase II and such polypeptides). It will be understood that specific antibodies may also interact with other proteins (for example, S. aureus protein A or other antibodies in ELISA techniques) through interactions with sequences outside the variable region of the antibodies, and in particular, in the constant region of the molecule. Screening assays to determine binding specificity of an antibody of the invention are well known and routinely practiced in the art. For a comprehensive discussion of such assays, see Harlow et al. (Eds), Antibodies A Laboratory Manual; Cold Spring Harbor Laboratory; Cold Spring Harbor, N.Y. (1988), Chapter 6. Antibodies that recognize and bind fragments of the heparanase II polypeptides of the invention are also contemplated, provided that the antibodies are, first and foremost, specific for heparanase II polypeptides. Antibodies of the invention can be produced using any method well known and routinely practiced in the art.

Non-human antibodies may be humanized by any methods known in the art. In one method, the non-human CDRs are inserted into a human antibody or consensus antibody framework sequence. Further changes can then be introduced into the antibody framework to modulate affinity or immunogenicity.

Antibodies of the invention are useful for, for example, therapeutic purposes (by modulating activity of heparanase II), diagnostic purposes to detect or quantitate heparanase II, as well as purification of heparanase II. Kits comprising an antibody of the invention for any of the purposes described herein are also comprehended. In general, a kit of the invention also includes a control antigen for which the antibody is immunospecific

The invention includes several assay systems for identifying heparanase II inhibitors. In solution assays, methods of the invention comprise the steps of (a) contacting a heparanase II polypeptide with one or more candidate inhibitor compounds and (b) identifying the compounds that inhibit the heparanase II enzymatic activity. Agents that modulate (i.e., increase, decrease) heparanase II activity or expression may be identified by incubating a putative modulator with a cell expressing a heparanase II polypeptide or polynucleotide and determining the effect of the putative modulator on heparanase II activity or expression. The selectivity of a compound that modulates the activity of heparanase II can be evaluated by comparing its effects on heparanase II to its effect on other heparanase enzymes. Modulators of heparanase II activity will be therapeutically useful in treatment of diseases and physiological conditions in which normal or aberrant heparanase II activity is involved.

The activity may be measured in a variety of ways. Haimovitz-Friedman et al. (Blood 78: 789-796, 1991) describe an assay for heparanase activity that involves the culturing of endothelial cells in radiolabeled ³⁵SO4 to produce radiolabeled heparan sulfate proteoglycans, the removal of the cells which leaves the deposited extracellular matrix that contains the ³⁵S-HSPG, the addition of potential sources of heparanase activity, and the detection of possible activity by passing the supernatant from the radiolabeled extracellular matrix over a gel filtration column and monitoring for changes of the size of the radiolabeled material that would indicate that HSPG degradation had taken place.

Nakajima et al. (Anal. Biochem. 196: 162-171, 1986) describe a solid-phase substrate for the assay of melanoma heparanase activity. Heparan sulfate from bovine lung is chemically radiolabeled by reacting it with [¹⁴C]-acetic anhydride. Free amino groups of the [¹⁴C]-heparan sulfate were acetylated and the reducing termini were aminated. The [¹⁴C]-heparan sulfate was chemically coupled to an agarose support via the introduced amine groups on the reducing termini. However, the usefulness of the Nakajima et al. assay is limited by the fact that the substrate is an extensively chemically modified form of naturally occurring heparan sulfate.

Khan and Newman (Anal. Biochem. 196: 373-376, 1991) describe an indirect assay for heparanase activity. In this assay, heparin is quantitated by its ability to interfere with the color development between a protein and the dye Coomassie brilliant blue. Heparanase activity is detected by the loss of this interference. This assay is limited in use for screening because it is so indirect that other non-heparin compounds could also interfere with the protein-dye reaction. It should be recognized, of course, that these assays are mentioned by way of example only and that these methods might be modified or that other methods of assaying heparanase activity would be apparent to one skilled in the art.

The invention also comprehends high throughput screening (HTS) assays to identify compounds that interact with or inhibit biological activity (i.e., inhibit enzymatic activity, binding activity, etc.) of a heparanase II polypeptide. HTS assays permit screening of large numbers of compounds in an efficient manner. Cell-based HTS systems are contemplated to investigate heparanase II enzyme-substrate interaction. HTS assays are designed to identify “hits” or “lead compounds” having the desired property, from which modifications can be designed to improve the desired property. Chemical modification of the “hit” or “lead compound” is often based on an identifiable structure/activity relationship between the “hit” and the heparanase II polypeptide.

Mutations in the heparanase II gene that result in loss of normal function of the heparanase II gene product underlie heparanase II -related human disease states. The invention comprehends gene therapy to restore heparanase II activity to treat those disease states. Delivery of a functional heparanase II gene to appropriate cells is effected ex vivo, in situ, or in vivo by use of vectors, and more particularly viral vectors (e.g., adenovirus, adeno-associated virus, or a retrovirus), or ex vivo by use of physical DNA transfer methods (e.g., liposomes or chemical treatments). See, for example, Anderson, Nature, supplement to vol. 392, no. 6679, pp.25-20 (1998). For additional reviews of gene therapy technology see Friedmann, Science, 244: 1275-1281 (1989); Verma, Scientific American: 68-84 (1990); and Miller, Nature, 357: 455-460 (1992). Alternatively, it is contemplated that in other human disease states, preventing the expression of or inhibiting the activity of heparanase II will be useful in treating the disease states. It is contemplated that antisense therapy or gene therapy could be applied to negatively regulate the expression of heparanase II.

Heparanase II is expressed at elevated levels in mobile invasive cells. Examples include invasive melanoma, lymphoma, mastocytoma, mammary adenocarcinoma, leukemia and rheumotoid fibroblasts providing an indication that aberrantly expressed heparanase II activity may correlate with metastasis. Inhibiting hepararanse II activity whether by small molecule inhibitor or by antibodies with specificity for provides a useful treatment modality for the prevention of metastasis.

Because heparanase breaks down the extracellular matrix with attendant release of growth factors, enzymes, and chemotactic proteins, an agent that inhibits heparanase activity should find therapeutic application in cancer, CNS and neurodegenerative diseases, inflammation, and in cardiovascular diseases such as restenosis following angioplasty and atherosclerosis.

The heparanase II of the present invention, both naturally and recombinantly produced, may be used for the same applications that have previously been for other heparanases. These applications include, but are not limited to, the acceleration of wound healing, the blocking of angiogenesis, and the degradation of heparin and the neutralization of heparin's anticoagulant properties during surgery, wherein an immobilized heparanase filter is connected to extracorporeal devices to degrade heparin and neutralize its anticoagulant properties during surgery. Immobilization onto filters can be achieved by methods well known in the art, such as those disclosed by Langer et al. (Biomateriais: Inter-facial Phenomenon and Applications, Cooper et al., eds., pp. 493-509 (1982)), and in U.S. Pat. Nos. 4,373,023, 4,863,611 and 5,211,850.

In addition to its application as a target for development of molecules that either enhance (increase) or inhibit (decrease) heparanase activity, the isolated heparanase of the subject invention can be used therapeutically for wound healing or as a means of blocking angiogenesis or inflammation. It can also be immobilized onto filters and used to degrade heparin from the blood of patients post-surgery.

Wound treatment can be achieved by administering to an afflicted individual an effective amount of a pharmaceutical composition comprising the isolated heparanase, or an agent that enhances heparanase activity, in combination with a pharmaceutically acceptable, preferably slow releasing, carrier. See, e.g., WO 91/02977, incorporated herein by reference.

Administration of heparanase for inhibition of angiogenesis can be localized or systemic depending upon the application; doses may vary as well. In treatment of psoriasis or diabetic retinopathy, the heparanase, or an agent capable of enhancing heparanase activity, is delivered in a topical carrier. Biodegradable polymeric implants may be used to deliver the heparanase for treatment of solid tumors. See, e.g., U.S. Pat. No. 5,567,417, incorporated herein by reference.

Heparanase, or an agent that enhances heparanase activity, can also be infused into the vasculature to block accumulation and diapedesis of neutrophils at sites of inflammation with or without added domains to confer selectivity in delivery. See, e.g., WO 9711684, incorporated herein by reference

Additional features of the invention will be apparent from the following Examples.

### EXAMPLE 1

### Cloning of Heparanase II

Genomic database mining of public and private databases Incyte [LifeSeq, LifeSeq FL, LifeSeq Assembled, LifeSeq Gold, and LifeSeq Atlas], GenBank, and the Institute for Genomic Research Total Human Consensus databases were performed using the BLAST search tool. Contig assemblies and Clustal W multiple sequence alignments are performed using the bioinformatics tools provided with the Incyte LifeSeq database interface.

Identification and full-length cloning of a heparanase paralg—Molecular definition of human platelet heparanase II was achieved using a combination of protein sequencing and mining of expressed sequence tag databases. The predicted amino acid sequence of human heparanase I was used to interogate the Incyte databases using the FASTA search tool. In addition to the ESTs displaying an exact match to the heparanase I sequence, three additional ESTs were detected. Each of these ESTs shows approximately 40% shared identity with the heparanase I amino acid sequence, consistent with a paralog relationship. These three EST sequences could not be assembled into a contig, indicating that either they are derived from non-overlapping regions of a single gene or they are derived from as many as three separate human genes. To resolve this issue, Incyte clones 1654352 (prostate tumor library), 3207353 (corpus cavernosum), and 3704980 (corpus cavernosum) were obtained and completely sequenced on both strands to provide 100% accurate sequence. Subsequent queries of the Incyte databases with these cDNA sequences and the BLAST search tool identified several additional EST matches. Incyte clones 3529440 (normal bladder) and 3385824 (normal esophagus) were also obtained and completely sequenced Additional 5′ DNA sequence was established by 5′ RACE analysis using a Marathon-ready cDNA template obtained from Clontech (Palo Alto, Calif.). An antisense primer specific for the shared 5′ region of cDNAs 3207353 or 3385824

[GGCAACATCACTTCGAACAATGTC] SEQ ID NO: 3 was paired with the universal AP-1 primer in the PCR on a Marathon-ready cDNA templates prepared from either human prostate, human small intestine, human bladder, or human heart RNA (Clontech, Palo Alto, Calif.). The following thermocycle parameters were used:
1 min @ 94° C.
30 sec @ 94° C., 4 min @ 72° C. for 5 cycles
30 sec @ 94° C., 4 min @ 70° C. for 5 cycles
30 sec @ 94° C., 4 min @ 68° C. for 25 cycles
10 min extension @ 72° C.

Specific amplification products were not detected by agarose gel analysis of the primary 5′ RACE products so a nested amplification was performed. The primary amplification products (5 μl) were diluted with 245 μl water and 5 μl of the resulting mixtures taken for nested amplification. Primer AP-2 (Clonetech, Palo Alto, Calif.) was paired with the nested primer specific for the 5′ end of clone 3207353 [CGAGCCAGCCATCATGMTGATG] SEQ ID NO:4 human prostate and human small intestine templates or specific for the 5′ end of clone 3385824 [GAGAGGAAAGGTTCCCAGGACAG] SEQ ID NO:5 human bladder and human heart templates and PCR amplification performed exactly as described above.

The contents from the PCR reactions were loaded onto a 1.2% agarose gel and electrophoresed. The DNA band of expected size was excised from the gel, placed in GenElute Agarose spin column (Supelco) and spun for 10 minutes at maximum speed in a Savant microcentrifuge. The eluted DNA was ethanol-precipitated and resuspended in 6 μl H2O for ligation.

The isolated PCR fragment containing the heparanase II coding sequences were ligated into a commercial vector using Invitrogen's Original TA Cloning Kit. The ligation reaction, which consisted of 6 μl DNA, 1μl 10× ligation buffer, 2 μl of plasmid pCR2.1 (25 ng/μl), Invitrogen), and 1 μl T4 DNA ligase (Invitrogen), was incubated overnight at 14° C. The reaction was heated at 65° C. for 10 minutes to inactivate the ligase enzyme, and then one microliter of the ligation reaction was transformed in One Shot cells (Invitrogen) and plated onto ampicillin plates. A single colony containing an insert was used to inoculate a 5 ml culture of LB medium. The culture was grown for 18 hours, and plasmid DNA from the culture was isolated using a Concert Rapid Plasmid Miniprep System (GibcoBRL) and sequenced to confirm that the plasmid contained the heparanase II insert.

Upon confirmation of the insert, the same transformant was used to inoculate a 50 ml culture of LB medium. The culture was grown for 16 hours at 37° C., and centrifuged into a cell pellet. Plasmid DNA was isolated from the pellet using a Qiagen Plasmid Midi Kit and again sequenced to confirm successful cloning of the heparanase II insert, using an ABI377 fluorescence-based sequencer (Perkin Elmer/Applied Biosystems Division, PE/ABD, Foster City, Calif.) and the ABI PRISMTM Ready Dye-Deoxy Terminator kit with Taq FSTM polymerase.

Each ABI cycle sequencing reaction contained about 0.5 μg of plasmid DNA. Cycle-sequencing was performed using an initial denaturation at 98° C. for 1 minute, followed by 50 cycles: 98° C. denaturation for 30 seconds, annealing at 50° C. for 30 seconds, and extension at 60° C. for 4 minutes. Temperature cycles and times were controlled by a Perkin-Elmer 9600 thermocycler. Extension products were isolated using Centriflex™ gel filtration cartridges (Advanced Genetic Technologies Corp., Gaithersburg, Md.). Each reaction product was loaded by pipette onto the column, which was then centrifuged in a swinging bucket centrifuge (Sorvall model RT6000B table top centrifuge) at 1500×g for 4 minutes at room temperature. Column-purified samples were dried under a vacuum for about 40 minutes and then dissolved in 5 μl of a DNA loading solution (83% deionized formamide, 8.3 mM EDTA, and 1.6 mg/ml Blue Dextran). The samples were then heated to 90° C. for three minutes and loaded into the gel sample wells for sequence analysis by the ABI377 sequencer. Sequence analysis was done by importing ABI377 files into the Sequencher® program (Gene Codes, Ann Arbor, Mich.). Generally sequence reads of 700 bp were obtained. Potential sequence errors were minimized by obtaining sequence information from both DNA strands and by re-sequencing difficult areas using primers at different locations until all sequencing ambiguities were removed.

It should be recognized that this method of obtaining the sequence of SEQ ID NO:1 is exemplary and that by disclosing SEQ ID NO:1 it provides one skilled in the art a multitude of methods of obtaining the entire sequence of SEQ ID NO:1. By way of example, it would be possible to generate probes from the sequence disclosed in SEQ ID NO:1 and screen human cDNA or genomic libraries and thereby obtain the entire SEQ ID NO:1 or its genomic equivalent. Sambrook, et al., (Eds.), Molecular Cloning: A Laboratory Manual, Cold Spring Harbor Laboratory Press: Cold Spring Harbor, N.Y. (1989). Also by way of example, one skilled in the art would immediately recognize that given the sequence disclosed in SEQ ID NO:1 it is then possible to generate the appropriate primers for PCR amplification to obtain the entire sequence represented by SEQ ID NO:1. (see e.g., PCR Technology, H. A. Erlich, ed., Stockton Press, New York, 1989; PCR Protocols: A Guide to Methods and Applications, M. A. Innis, David H. Gelfand, John J. Sninsky, and Thomas J. White, eds., Academic Press, Inc., New York, 1990,

### EXAMPLE 2

### Analysis of the Heparanase II Sequence

Computer-aided analysis of the predicted heparanase II amino acid sequence of the predicted amino acid sequence of human heparanase II was analyzed for various protein motifs using both the ProSite dictionary and the Pfam database as well as using prediction methods available on the Center for Biotechnology Sequence Analysis (CBS) server in the Biotechnology Department at the University of Denmark. The ProSite motifs analysis identified canonical acceptor sites for Asn-linked glycosylation [alignment positions 217 and 334] and consensus acceptor sites for phosphorylation by protein kinase C [alignment positions 66, 97, 98, 449, 458]. Also, potential sites for C-terminal amidation [G-R/K-R/K] were localized to alignment positions 116 and 315. The heparanase II amino acid sequence was analyzed for the presence of a signal sequence using the SignalP neural net-based prediction. method available on the CBS server. Using neural nets trained on eukaryotic signal sequences, the first 41 NH2-terminal amino acid residues are predicted to be a signal peptide based on all four parameters and the most likely site of cleavage is between positions 41 and 42 [SQA↓GD]. No predicted transmembrane domains were detected in the human heparanase II sequence. The presence of a signal sequence and the lack of predicted transmembrane segments are consistent with heparanase II being a secreted protein. The positions of these various functional motifs in the heparanase II amino acid sequence are summarized in FIG. 1.

The sequence of human heparanase II was then aligned with the predicted sequence of heparanase I using the Clustal W algorithm and the results are shown in FIG. 2. Heparanase I and II display 43% shared identity at the amino acid sequence level with 213 identical residues.

Both heparanase I and heparanase II share a similar domain organization including a relatively long signal peptide followed by a catalytic domain that lacks predicted transmembrane segments. This organization is consistent with both heparanases I and II being secreted proteins. Motifs shared by both polypeptides include canonical acceptor sites for N-linked glycosylation, phosphorylation by protein kinase C, and for C-terminal amidation. The consensus sites for tyrosine phosphorylation and PKA in heparanase I are not present in heparanase II.

The predicted amino acid sequence of heparanase II does not show significant identity to any protein in the November 1999 release of SwissProt, except heparanase I. The availability of two related polypeptide sequences with little homology to other known proteins allows predictions to be made regarding structure-function. Assuming that the heparanase I and 11 genes arose by duplication and subsequent divergence of a single ancestoral gene, regions of the polypeptide sequence important for function are likely to be conserved.

In several of the reports describing heparanase I, the enzyme had been characterized as a 50 kDa single chain peptide. Vladavsky, I et al. Nature Genetics 5:793-802, 1999, Hulet et al. Nature Genetics 5:803-809, 1999, Toyoshima, M. et al. J. Biol. Chem. 274:24153-60, 1999, Kussie, P. H. et al. Biochem. Biophys. Res. Comm. 261:183-187, 1999. Fairbanks et al, however, recently presented evidence that heparanase I is initially synthesized as pro-heparanase I that is proteolytically processed into a two chain heterodimer. Fairbanks et al. J. Biol. Chem. 274(42):29587-90, 1999. Alignment of the human heparanase I and human heparanase II amino acid sequences (FIG. 2) reveals that the processing sites are partially conserved. The processing sites in heparanase I involve the excision of a 44 or 45 amino acid region near the N-terminus by sequential proteolytic cleavage at the sequence PKK↓EST or PKKE↓ST and HYQ↓KKF to generate the N-terminus and C-terminus of the excised peptide, respectively. By alignment, processing sites in heparanase II are NLR↓NPA and DKQ↓KGC, indicating conservative substitutions in the N-terminal P1/P1′ positions and identical P1/P1′ residues at the C-terminal processing site.

Examination of the enzymatic activity of native platelet heparanase I has revealed that the enzyme is an endo-β-glucuronidase. The enzyme preferentially cleaves heparan sulfate between D-glucuronic acid and N-acetylglucosamine residues in which the uronic acid on the reducing side of the N-acetylglucosamine is O-sulfated. Glycosidases function by two general mechanisms resulting in either retention or inversion of configuration at the hydrolysis site. In both cases, two acidic amino acids, usually glutamic acids, are directly involved in catalysis. The acidic side chain of one amino acid serves as the nucleophile while the other acts as a general acid/general base in the reaction mechanism. Structure-function studies of lysosomal human exo-β-glucuronidase involved in the degradation of glycosaminoglycans implicates a pair of glutamic acid residues (Glu⁴⁵¹ and Glu⁵⁴⁰) in the catalytic mechanism. Alternatively, the catalytic pair in lysozyme involves Glu35 and Asp52. Taken together, these results suggests that a pair of conserved amino acid residues with acidic side chains in heparanase I and II may participate in the endo-β-glucuronidase activity of both enzymes. Inspection of the Clustal W alignment of the heparanase I and II amino acid sequences revealed 15 aspartic acid residues that are conserved between the two sequences but no glutamic acid residues. Six of these aspartic acid residues are nested in clusters of sequence identity that involve >75% identity over >15 amino acid residues. One or more of these regions are likely to contribute the residues involved in heparanase catalysis.

### EXAMPLE 3

### Hybridization Analysis Demonstrates that Heparanase II is Expressed in Bladder, Prostate, Stomach, Small Intestine, Uterus and Brain

The tissue distribution of expression of human heparanase II was established by Northern blot. For Northern analysis, heparanase II transcripts were visualized using a cDNA probe derived from Incyte clone 3704980 and the results are shown in FIG. 3. A single 4.4 kb transcript was detected at the highest level in bladder and lower amounts were also present in prostate, stomach, small intestine, uterus and brain. No signal was detected in skeletal muscle, colon, heart, thymus, spleen, kidney, liver, placenta, lung, or peripheral blood leukocytes under these conditions (data not shown). Common sources of heparanase activity include human platelets, placenta, and tumor cell lines and the enzyme from both platelets and tumor cell lines are biochemically indistinguishable. Indeed, Northern blot analysis of the human tissue distribution of expression of heparanase I revealed high expression levels in placenta and peripheral blood leukocytes and somewhat reduced levels in spleen, lymph node, bone marrow and fetal liver. We could not detect the expression of heparanase II in placenta, peripheral blood leukocytes (data not shown) but rather observed the highest level of expression in tissues rich in vascular smooth muscle (FIG. 3). A survey of the expression pattern of heparanase enzyme activity has identified vascular smooth muscle cells as a source of activity. These results indicate that heparanase I and II have a non-overlapping expression pattern in human tissues and each may serve tissue-specific functional roles. Perhaps the substrate specificity for heparan sulfate hydrolysis is distinct between these two isozymes and the work reported here enables the preparation of recombinant heparanase II for further characterization.

### EXAMPLE 4

### Expression of Heparanase II in Eukaryotic Host Cells

To produce heparanase II protein, a heparanase II-encoding polynucleotide is expressed in a suitable host cell using a suitable expression vector, using standard genetic engineering techniques. For example, the heparanase II-encoding sequences described in Example 1 are subcloned into the commercial expression vector pzeoSV2 (Invitrogen, San Diego, Calif.) and transfected into Chinese Hamster Ovary (CHO) cells using the transfection reagent fuGENE 6 (Boehringer-Mannheim) and the transfection protocol provided in the product insert. Other eukcryotic cell lines, including human embryonic kidney HEK 293 and COS cells, are suitable as well. Cells stably expressing heparanase II are selected by growth in the presence of 100 μg/ml zeocin (Stratagene, LaJolla, Calif.). Optionally, the heparanase II is isolated from the cells using standard chromatographic techniques. To facilitate purification, antisera is raised against one or more synthetic peptide sequences that correspond to portions of the heparanase II amino acid sequence, and the antisera is used to affinity purify heparanase II. The heparanase II also may be expressed in frame with a tag sequence (e.g., polyhistidine, hemaggluttinin, FLAG) to facilitate purification. Moreover, it will be appreciated that many of the uses for heparanase II polypeptides, such as assays described below, do not require purification of heparanase II from the host cell.

### EXAMPLE 5

### Antibodies to Heparanase II

Standard techniques are employed to generate polyclonal or monoclonal antibodies to the heparanase II enzyme, and to generate useful antigen-binding fragments thereof or variants thereof, including “humanized” variants. Such protocols can be found, for example, in Sambrook et al., Molecular Cloning: a Laboratory Manual. Second Edition, Cold Spring Harbor, N.Y.: Cold Spring Harbor Laboratory (1989); Harlow et al. (Eds), Antibodies A Laboratory Manual; Cold Spring Harbor Laboratory; Cold Spring Harbor, N.Y. (1988); and other documents cited below. In one embodiment, recombinant heparanase II polypeptides (or cells or cell membranes containing such polypeptides) are used as antigen to generate the antibodies. In another embodiment, one or more peptides having amino acid sequences corresponding to an immunogenic portion of heparanase II (e.g., 6, 7, 8, 9, 10, 11, 12, 13, 14, 15, 16, 17, 18, 19, 20, or more amino acids) are used as antigen In order to mimic a protein epitope with a small synthetic peptide, it is important to choose a sequence that is hydrophilic, surface-oriented, and flexible. Van Regenmortel, 1986. This is because most naturally occurring proteins found in physiological solutions have their hydrophilic residues on the surface and their hydrophobic residues buried. Antibodies generally bind to epitopes on the surfaces of naturally occurring proteins. Several known epitopes have a high degree of mobility The N- and C-termini of proteins are generally surface-oriented since they contain charged groups, i.e., NH₃⁺ and COO⁻. They often have a high degree of mobility as well, since they are located at the ends. These termini are often chosen as candidates for synthesis because they possess all three properties. Peptides corresponding to surface residues of heparanase II, especially hydrophilic portions are contemplated. Also contemplated are peptides located at the amino and carboxy terminal ends of heparanase II

Algorithms have been developed to assign values of hydrophilicity, surface accessibility, and flexibility to each amino acid residue within a given protein sequence. The same has been done to assign an antigenic index to each residue, giving an indication of how antigenic that residue is within a specific sequence. Hopp and Woods, Mol. Immunol, 1983 20(4): p. 483-9, Hopp and Woods, Proc. Natl Acad. Sci USA 1981, 78(6) p. 3824-8. Although selection of hydrophilic segments has been widely used in generating anti-peptide antibodies that are useful for binding native antigen. Unlike antibodies however, T cell receptors see relatively small segments of protein antigen after cleavage and unfolding. T cell antigenic sites has also been addressed by predictive computer models. Margalit, H. et al., J. Immunol. 1987 138(7): pg 2213-29

Computer programs useful for the prediction of epitopes are commercially available. For example MacVector® (Oxford Molecular, Oxford, UK) and Protean® (DNAStar Madison, Wis. 53715) Once a peptide antigen is selected and synthesized the antigen may be mixed with an adjuvant or linked to a hapten to increase antibody production.

A. Polyclonal or Monoclonal antibodies

As one exemplary protocol, recombinant heparanase II or a synthetic fragment thereof is used to immunize a mouse for generation of monoclonal antibodies (or larger mammal, such as a rabbit, for polyclonal antibodies). To increase antigenicity, peptides are conjugated to Keyhole Lympet Hemocyanine (Pierce), according to the manufacturer's recommendations. For an initial injection, the antigen is emulsified with Freund's Complete Adjuvant and injected subcutaneously. At intervals of two to three weeks, additional aliquots of heparanase II antigen are emulsified with Freund's Incomplete Adjuvant and injected subcutaneously. Prior to the final booster injection, a serum sample is taken from the immunized mice and assayed by western blot to confirm the presence of antibodies that immunoreact with heparanase II. Serum from the immunized animals may be used as a polyclonal antisera or used to isolate polyclonal antibodies that recognize heparanase II. Alternatively, the mice are sacrificed and their spleen removed for generation of monoclonal antibodies.

To generate monoclonal antibodies, the spleens are placed in 10 ml serum-free RPMI 1640, and single cell suspensions are formed by grinding the spleens in serum-free RPMI 1640, supplemented with 2 mM L-glutamine, 1 mM sodium pyruvate, 100 units/ml penicillin, and 100 μg/ml streptomycin (RPMI) (Gibco, Canada). The cell suspensions are filtered and washed by centrifugation and resuspended in serum-free RPMI. Thymocytes taken from three naive Balb/c mice are prepared in a similar manner and used as a Feeder Layer. NS-1 myeloma cells, kept in log phase in RPMI with 10% fetal bovine serum (FBS) (Hyclone Laboratories, Inc., Logan, Utah) for three days prior to fusion, are centrifuged and washed as well.

To produce hybridoma fusions, spleen cells from the immunized mice are combined with NS-1 cells and centrifuged, and the supernatant is aspirated. The cell pellet is dislodged by tapping the tube, and 2 ml of 37° C. PEG 1500 (50% in 75mM Hepes, pH 8.0) (Boehringer Mannheim) is stirred into the pellet, followed by the addition of serum-free RPMI. Thereafter, the cells are centrifuged and resuspended in RPMI containing 15% FBS, 100 μM sodium hypoxanthine, 0.4 μM aminopterin, 16 μM thymidine (HAT) (Gibco), 25 units/ml IL-6 (Boehringer Mannheim) and 1.5×106 thymocytes/ml and plated into 10 Corning flat-bottom 96-well tissue culture plates (Corning, Corning N.Y.).

On days 2, 4, and 6, after the fusion, 100 μl of medium is removed from the wells of the fusion plates and replaced with fresh medium. On day 8, the fusions are screened by ELISA, testing for the presence of mouse IgG that binds to heparanase II. Selected fusion wells are further cloned by dilution until monoclonal cultures producing anti-heparanase II antibodies are obtained.

B. Humanization of anti-heparanase II monoclonal antibodies

The expression pattern of heparanase II as reported herein and the proven track record of GPCR's as targets for therapeutic intervention suggest therapeutic indications for heparanase II inhibitors (antagonists). Heparanase l-neutralizing antibodies comprise one class of therapeutics useful as heparanase II antagonists. Following are protocols to improve the utility of anti-heparanase II monoclonal antibodies as therapeutics in humans, by “humanizing” the monoclonal antibodies to improve their serum half-life and render them less immunogenic in human hosts (i.e., to prevent human antibody response to non-human anti-heparanase II antibodies).

The principles of humanization have been described in the literature and are facilitated by the modular arrangement of antibody proteins. To minimize the possibility of binding complement, a humanized antibody of the IgG4 isotype is contemplated by the invention.

For example, a level of humanization is achieved by generating chimeric antibodies comprising the variable domains of non-human antibody proteins of interest with the constant domains of human antibody molecules. (See, e.g., Morrison and Oi, Adv. Immunol., 44:65-92 (1989). The variable domains of heparanase II neutralizing anti-heparanase II antibodies are cloned from the genomic DNA of a B-cell hybridoma or from cDNA generated from mRNA isolated from the hybridoma of interest. The V region gene fragments are linked to exons encoding human antibody constant domains, and the resultant construct is expressed in suitable mammalian host cells (e.g., myeloma or CHO cells).

To achieve an even greater level of humanization, only those portions of the variable region gene fragments that encode antigen-binding complementarity determining regions (“CDR”) of the non-human monoclonal antibody genes are cloned into human antibody sequences. [See, e.g., Jones et al., Nature, 321:522-525 (1986); Riechmann et al., Nature, 332:323-327 (1988); Verhoeyen et al., Science, 239:1534-36 (1988); and Tempest et al., Bio/Technology, 9:266-71 (1991). If necessary, the β-sheet framework of the human antibody surrounding the CDR3 regions also is modified to more closely mirror the three dimensional structure of the antigen-binding domain of the original monoclonal antibody. (See Kettleborough et al., Protein Engin., 4:773-783 (1991); and Foote et al., J. Mol. Biol., 224:487-499 (1992).)

In an alternative approach, the surface of a non-human monoclonal antibody of interest is humanized by altering selected surface residues of the non-human antibody, e.g., by site-directed mutagenesis, while retaining all of the interior and contacting residues of the non-human antibody. See Padlan, Molecular Immunol., 28(4/5):489-98 (1991).

The foregoing approaches are employed using heparanase 11-neutralizing anti-heparanaase II monoclonal antibodies and the hybridomas that produce them to generate humanized heparanase II-neutralizing antibodies useful as therapeutics to treat or palliate conditions wherein heparanase II expression or ligand-mediated heparanase II signaling is detrimental.

C. Human Heparanase II-Neutralizing Antibodies from Phage Display

Human heparanase II-neutralizing antibodies are generated by phage display techniques such as those described in Aujame et al., Human Antibodies, 8(4):155-168 (1997); Hoogenboom, TIBTECH, 15:62-70 (1997); and Rader et al., Curr. Opin. Biotechnol., 8:503-508 (1997), all of which are incorporated by reference. For example, antibody variable regions in the form of Fab fragments or linked single chain Fv fragments are fused to the amino terminus of filamentous phage minor coat protein pill. Expression of the fusion protein and incorporation thereof into the mature phage coat results in phage particles that present an antibody on their surface and contain the genetic material encoding the antibody. A phage library comprising such constructs is expressed in bacteria, and the library is panned (screened) for heparanase II-specific phage-antibodies using labelled or immobilized heparanase II as antigen-probe.

D. Human Heparanase II-Neutralizing Antibodies from Transgenic Mice

Human heparanase II-neutralizing antibodies are generated in transgenic mice essentially as described in Bruggemann and Neuberger, Immunol. Today, 17(8):391-97 (1996) and Bruggemann and Taussig, Curr. Opin. Biotechnol., 8:455-58 (1997). Transgenic mice carrying human V-gene segments in germline configuration and that express these transgenes in their lymphoid tissue are immunized with a heparanase II composition using conventional immunization protocols. Hybridomas are generated using B cells from the immunized mice using conventional protocols and screened to identify hybridomas secreting anti-heparanase II human antibodies (e.g., as described above).

### EXAMPLE 6

### Assay for Heparanase Activity

Preparation of 35S-HSPG (>70 K) for use in the heparanase assay: 35S-HSPG (>70 K) is prepared from mice bearing a basement membrane tumor that overproduces HSPG (EHS tumor), using modifications of the method of Ledbetter et al. (Biochemistry 26: 988-995 (1987)). Briefly, the radiolabeled HSPG is prepared by injecting C57BL mice bearing the EHS tumor with sodium [35S] sulfate (0.5 mCi/mouse) 18 h before harvesting the tumor. The HSPG is extracted from the weighed tumor with 6 volumes (w/v) of Buffer A (3.4 M NaCl, 0.1 M 6-aminohexanoic acid, 0.04 M EDTA, 0.008 M N-ethylmaleimide, 0.002 M PMSF, and 0.05 M Tris-HCl, pH 6.8), by homogenization with a Polytron for 30 s, followed by stirring at 4° C. for 1 h. Insoluble material is collected by centrifugation (12,000×g for 10 min), and the supernatant is discarded. The insoluble residue is reextracted with 2 volumes (original tumor weight) of Buffer A for 30 min with stirring at 4° C. Insoluble material was again collected by centrifugation, and the supernatant fraction was discarded. The insoluble material is then suspended in 6 volumes of Buffer B (6 M urea, 0.1 M 6-aminohexanoic acid, 0.04 M EDTA, 0.002 M PMSF, and 0.05 M Tris-HCl, pH 6.8), homogenized with an electric homogenizer (Polytron) for 30 s, and stirred for 2 h at 4° C. The mixture is centrifuged to remove insoluble material, and the supernatant was retained. The insoluble material is reextracted with 2 volumes of Buffer B. The mixture is centrifuged, and the supernatant is combined with the previous supernatant.

35S-HSPG is isolated from the Buffer B supernatant by sequential chromatography on anion exchange and gel filtration columns. The Buffer B supernatant is dialyzed overnight against 10 volumes of 6 M urea, 0.15 M NaCl, 0.05 M Tris-HCl, pH 6.8, and is adjusted to contain 0.5% non-ionic detergent (triton X-100). This supernatant (from 11 g tumor) is chromatographed on a 30 ml column of anion exchange resin (DEAE-Sephacel) equilibrated with 6 M urea, 0.15 M NaCl, 0.05% Triton X-100, 0.05 M Tris-HCl, pH 6.8. After loading the supernatant and washing with the equilibration buffer, the column is developed with a 250 ml linear gradient between 0.15 M NaCl and 1.15 M NaCl (flow =2.0 ml/min). Fractions are sampled for radioactivity, and those containing the 35SO4 label that eluted from the DEAE-Sephacel between 0.4 M and 0.8 M NaCl are pooled. The proteoglycan is precipitated by the addition of 4 volumes of 100% EtOH at −20° C. overnight. The precipitate is collected by centrifugation and was solubilized in 1 ml of Buffer C (4 M Gu-HCl, 20 mM Tris-HCl, pH 7.2). This solubilized pellet is used for chromatography on a calibrated gel filtration column (1.0×50 cm column of Superose 6; Pharmacia) equilibrated in Buffer C (Flow=0.5 ml/min). Fractions are sampled for radioactivity, and those containing the 35S04 label that elutes with a molecular weight 70 kDa are pooled. The proteoglycan is precipitated with 100% EtOH as described above. The pellet is dissolved in 3 ml PBS, and dialyzed against 3×100 volumes of PBS. Each preparation of 35S-HSPG is confirmed to be 98% heparan sulfate by susceptibility to low pH nitrous acid degradation (Shiveley and Conrad, Biochemistry 15: 3932-3942 (1976)).

Measurement of Heparanase activity.—Heparanase activity from platelets or column fractions is detected by its ability to digest the 70 kDa 35S-HSPG to produce lower molecular weight products. not retained by a 30,000 MW cut-off membrane. Each digest contained 5-10 μl of sample to be assayed, 35S-HSPG (2000 cpm), 0.15 M NaCl, 0.03% human serum albumin, 10 μM MgCl2, 10 μM CaCl2, and 0.05 M Na acetate, pH 5.6 in a total volume of 300 μl. In the case of highly purified enzyme, the assay mixtures contain 2-5 ng of protein. Digests are carried out for 3 to 21 h. The presence of lower molecular weight radiolabeled products is detected by centrifugation through 30,000 MW-cutoff filters. The digests containing 2000 cpm of 35S-HSPG (>70 K) are centrifuged through 30,000 molecular weight cut-off filters (Millipore Ultrafree-MC 30,000 NMWL filter units). 35S-HSPG degradation is evident by the presence of radioactivity in the filtrate that passed through the 30 K membrane; this heparanase activity is expressed as the percent of total cpm<30,000 MW for a given digest. Analysis of heparan sulfate degradation by this method is quick and reproducible. One unit of heparanase II activity is defined as that amount of enzyme which produces 1% of the total starting cpm that can pass through the 30,000 MW cut-off membrane in one hour. For pH optimum determination, the 0.1 M Na acetate buffer is replaced by 50 mM citrate, citrate-phosphate, or phosphate buffer at varying pH's.

### EXAMPLE 7

### Assays to Identify Inhibitors of Heparanase II

The isolated heparanase II of the present invention, allows for the convenient selection of compounds having anti-heparanase II activity, i.e., inhibitors of heparanase activity (IHA), by measuring inhibition of heparanase II activity. Inhibition of heparanase activity can be measured by blocking heparanase II-mediated release of radioactive fragments from in vivo radiolabeled (HSPG)/heparin, as seen by the failure to produce breakdown fragments of a size that will pass through a 30,000 MW cut-off membrane. In this experiment, the ligand is radiolabeled to high specific activity by intraperitoneal injection of 0.5 mCi of 35S-sulfate into C57 mice bearing a 1-2 cm basement membrane tumor (EHS; Engelbreth, Holm, Swarm tumor). The tumor was harvested after 16 hours and the HSPG extracted in 4 volumes of 6 M urea, 20 mM Tris, pH 6.8, protease inhibitors, 0.15 M NaCl and 0.5% triton X-100. The urea extract was chromatographed on an anion exchange column and the HSPG eluted in a linear gradient of NaCl. The radiolabeled HSPG was exchanged into a solution of 4.0 M guanidine-HCl, 20 mM Tris, pH 7.4 and applied to a size exclusion column. The HSPG peak was pooled and exchanged into 0.15 mM NaCl and 20 mM Tris pH 7.4.

For purposes of high throughput screening, it is desirable to exploit assays that can be conducted in a 96-well microtiter plate format. In this case, the protein component of chromatographically purified 35S-HSPG is digested enzymatically by any non-specific enzyme, such as papain, to give free N-terminal amino groups. The [35 SO4] heparan sulfated peptides are then coupled to cyanogen bromide activated Sepharose-6B (Pharmacia Biotech) according to manufacturer's instructions. The 35S-Heparan sulfate-Sepharose 6B is resuspended in: 0.15 M NaCl, 0.03% human serum albumin, 10 pM MgCl2, 10 pM CaCl2, antiproteolytic agents (1 μg/ml leupeptin, 2 μg/ml antipain, 10 μg/ml benzamidine, 10 units/ml aprotinin, 1 μg/ml chymostatin, and 1 μg/ml pepstatin), and 0.05 M Na acetate, pH 5.6 and 5,000 cpm, in a total volume of 200 μl. This solution is then aliquoted into each well of a 96 well plate, which contains in each well a different test agent. Heparanase II (5 units) is added to each well, and the digestion is allowed to proceed overnight (16 h) at 37° C.

The digested products are then separated from the supernatant by centrifugation of the 96 well plate through a 30,000 MW cut-off membrane. The supernatant, containing cleaved heparan sulfate, is decanted and quantitated by scintillation counting. Agents which alter the activity of the heparanase II may thus be identified by comparing the amount of cleaved heparan sulfate in each test agent well with that in a control well lacking a test agent.

Additional features and variations of the invention will be apparent to those skilled in the art from the entirety of this application, including the detailed description, and all such features are intended as aspects of the invention. Likewise, features of the invention described herein can be re-combined into additional embodiments that also are intended as aspects of the invention, irrespective of whether the combination of features is specifically mentioned above as an aspect or embodiment of the invention. Also, only such limitations which are described herein as critical to the invention should be viewed as such; variations of the invention lacking limitations which have not been described herein as critical are intended as aspects of the invention.

It will be clear that the invention may be practiced otherwise than as particularly described in the foregoing description and examples.

Numerous modifications and variations of the present invention are possible in light of the above teachings and, therefore, are within the scope of the invention.

The entire disclosure of all publications cited herein are hereby incorporated by reference.

1. An isolated nucleic acid molecule comprising a polynucleotide with a nucleotide sequence selected from the group consisting of:
(a) a nucleotide sequence encoding a heparanase II polypeptide comprising the amino acid sequence of SEQ ID NO:2;
(b) a nucleotide sequence encoding a heparanase II polypeptide comprising the amino acid sequence at residues 42 through 534 of SEQ ID NO:2;
(c) a nucleotide sequence encoding a heparanase II polypeptide comprising the amino acid sequence at residues 42 through 129 of SEQ ID NO:2;
(d) a nucleotide sequence encoding a heparanase II polypeptide comprising the amino acid sequence at residues 42 through 161 of SEQ ID NO:2;
(e) a nucleotide sequence encoding a heparanase II polypeptide comprising the amino acid sequence at residues 130 through 534 of SEQ ID NO:2;
(f) a nucleotide sequence encoding the heparanase II polypeptide comprising the amino acid sequence at residues 162 through 534 of SEQ ID NO:2; and
(g) a nucleotide sequence that is complementary to any of the nucleotide sequences of (a), (b), (c), (d), (e) or (f).

2. The isolated nucleic acid molecule of claim 1, wherein the polynucleotide molecule of 1(a) comprises the nucleotide sequence at nucleotide positions 25 through 1626 of SEQ ID NO:1, the polynucleotide molecule of 1(b) comprises the nucleotide sequence at nucleotide positions 148 through 1626 of SEQ ID NO: 1, the polynucleotide molecule of 1(c) comprises the nucleotide sequence at nucleotide positions 148 through 411 of SEQ ID NO:1, the polynucleotide molecule of 1(d) comprises the nucleotide sequence at nucleotide positions 148 through 507 of SEQ ID NO:1, the polynucleotide molecule of 1(e) comprises the nucleotide sequence at nucleotide positions 412 through 1626 of SEQ ID NO:1 and the polynucleotide molecule of 1(f) comprises the nucleotide sequence at nucleotide positions 508 through 1626 of SEQ ID NO:1.

3. The isolated nucleic acid molecule of claim 1 wherein the polynucleotide molecule comprises a nucleotide sequence encoding a heparanase II polypeptide comprising the amino acid sequence of SEQ ID NO:2.

4. The isolated nucleic acid molecule of claim 1 wherein the polynucleotide molecule comprises a nucleotide sequence encoding a heparanase II polypeptide comprising the amino acid sequence at residues 42 through 534 of SEQ ID NO:2.

5. The isolated nucleic acid molecule of claim 1 wherein the polynucleotide molecule comprises a nucleotide sequence encoding a heparanase II polypeptide comprising the amino acid sequence at residues 42 through 129 of SEQ ID NO:2.

6. The isolated nucleic acid molecule of claim 1 wherein the polynucleotide molecule comprises a nucleotide sequence encoding a heparanase II polypeptide comprising the amino acid sequence at residues 42 through 161 of SEQ ID NO:2.

7. The isolated nucleic acid molecule of claim 1 wherein the polynucleotide molecule comprises a nucleotide sequence encoding a heparanase II polypeptide comprising the amino acid sequence at residues 130 through 534 of SEQ ID NO:2.

8. The isolated nucleic acid molecule of claim 1 wherein the polynucleotide molecule comprises a nucleotide sequence encoding a heparanase II polypeptide comprising the amino acid sequence at residues 162 through 534 of SEQ ID NO:2.

9. The isolated nucleic acid molecule of claim 1, wherein the polynucleotide molecule comprises the nucleotide sequence at nucleotide positions 25 through 1626 of SEQ ID NO:1.

10. The isolated nucleic acid molecule of claim 1, wherein the polynucleotide molecule comprises the nucleotide sequence at nucleotide positions 148 through 1626 of SEQ ID NO:1.

11. The isolated nucleic acid molecule of claim 1, wherein the polynucleotide molecule comprises the nucleotide sequence at nucleotide positions 148 through 411 of SEQ ID NO:1.

12. The isolated nucleic acid molecule of claim 1, wherein the polynucleotide molecule comprises the nucleotide sequence at nucleotide positions 148 through 507 of SEQ ID NO:1.

13. The isolated nucleic acid molecule of claim 1, wherein the polynucleotide molecule comprises the nucleotide sequence at nucleotide positions 412 through 1626 of SEQ ID NO:1.

14. The isolated nucleic acid molecule of claim 1, wherein the polynucleotide molecule comprises the nucleotide sequence at nucleotide positions 508 through 1626 of SEQ ID NO:1.

15. A vector comprising a nucleic acid molecule described in claim 1.

16 The vector of claim 15, wherein said nucleic acid molecule is operably linked to a promoter for the expression of a human heparanase polypeptide.

17. A host cell comprising the vector of claim 15.

18. The host cell of claim 17, wherein said host is a eukaryotic host.

19. The host cell of claim 17, wherein said host cell is a baculovirus cell.

20. A method of obtaining a human heparanase polypeptide comprising culturing the host cell of claim 17 and isolating said human heparanase polypeptide.

21. An isolated human heparanase polypeptide comprising an amino acid sequence selected from the group consisting of:
(a) an amino acid sequence of a heparanase II polypeptide comprising the amino acid sequence of SEQ ID NO:2; and
(b) a heparanase II polypeptide comprising the amino acid sequence at residues 42 through 534 of SEQ ID NO:2.

22. The isolated heparanase II polypeptide of claim 21 wherein the polypeptide molecule comprises the amino acid sequence of SEQ ID NO:2.

23. The isolated heparanase II polypeptide of claim 21 wherein the polypeptide molecule comprises the amino acid sequence at residues 42 through 534 of SEQ ID NO:2.

24. An isolated heparanase II enzyme consisting essentially of:
(a) an isolated heparanase II polypeptide comprising the amino acid sequence at residues 42 through 129 of SEQ ID NO: 2; and
(b) and isolated heparanase II polypeptide comprising the amino acid sequence at residues 130 through 534 of SEQ ID NO:2.

25. An isolated heparanase II enzyme consisting essentially of:
(a) an isolated heparanase polypeptide comprising the amino acid sequence at residues 42 through 161 of SEQ ID NO: 2; and
(b) and isolated heparanase polypeptide comprising the amino acid sequence at residues 162 through 534 of SEQ ID NO:2.

26. A heparanase II enzyme consisting essentially of:
(a) an isolated heparanase polypeptide comprising the amino acid sequence at residues 42 through 129 of SEQ ID NO: 2; and
(b) and isolated heparanase polypeptide comprising the amino acid sequence at residues 162 through 534 of SEQ ID NO:2

27. An antibody specific for the heparanase II polypeptide of claims.

28. A method for the identification of an agent that alters heparanase activity, said method comprising:
(a) determining the activity of the isolated heparanase II polypeptide
(i) in the presence of a test agent; and
(ii) in the absence of said test agent; and
(b) comparing the heparanase activity determined in step (a)(i) to the heparanase activity determined in step (a)(ii);
whereby a change in heparanase activity in sample (a)(i) has compared to sample (a)(ii) indicates that said agent alters the activity of said heparanase II polypeptide.

29. The method of claim 28, wherein said agent increases heparanase activity.

30. The method of claim 28, wherein said agent inhibits heparanase activity.

31. The method of claim 28, wherein the determination of heparanase activity is made by measuring the amount of radiolabeled heparin/heparan sulfate that is digested by said human heparanase enzyme.

32. A method for treating a disease state comprising the step of administering to a mammal in need of such treatment an amount of an antibody according to claim 27 sufficient to inhibit heparanase II enzymatic activity in the tissues of said mammal.

33. A method for treating a disease state comprising the step of administering to a mammal in need of such treatment an amount of an agent sufficient to alter heparanase II enzymatic activity in the tissues of said mammal.


SEQUENCE LISTING (6 sequences)

SEQ ID NO: 1 - DNA, 2326 bp, Homo sapiens
caggttttaaatcagagggattgaatgagggtgctttgtgccttccctgaagccatgccctccagcaactcccgcccccccgcgtgcctagccccgggggctctctacttggctctgttgctccatctctccctttcctcccaggctggagacaggagacccttgcctgtagacagagctgcaggtttgaaggaaaagaccctgattctacttgatgtgagcaccaagaacccagtcaggacagtcaatgagaacttcctctctctgcagctggatccgtccatcattcatgatggctggctcgatttcctaagctccaagcgcttggtgaccctggcccggggactttcgcccgcctttctgcgcttcgggggcaaaaggaccgacttcctgcagttccagaacctgaggaacccggcgaaaagccgcgggggcccgggcccggattactatctcaaaaactatgaggatgacattgttcgaagtgatgttgccttagataaacagaaaggctgcaagattgcccagcaccctgatgttatgctggagctccaaagggagaaggcagctcagatgcatctggttcttctaaaggagcaattctccaatacttacagtaatctcatattaacagagccaaataactatcggaccatgcatggccgggcagtaaatggcagccagttgggaaaggattacatccagctgaagagcctgttgcagcccatccggatttattccagagccagcttatatggccctaatattgggcggccgaggaagaatgtcatcgccctcctagatggattcatgaaggtggcaggaagtacagtagatgcagttacctggcaacattgctacattgatggccgggtggtcaaggtgatggacttcctgaaaactcgcctgttagacacactctctgaccagattaggaaaattcagaaagtggttaatacatacactccaggaaagaagatttggcttgaaggtgtggtgaccacctcagctggaggcacaaacaatctatccgattcctatgctgcaggattcttatggttgaacactttaggaatgctggccaatcagggcattgatgtcgtgatacggcactcattttttgaccatggatacaatcacctcgtggaccagaattttaacccattaccagactactggctctctctcctctacaagcgcctgatcggccccaaagtcttggctgtgcatgtggctgggctccagcggaagccacggcctggccgagtgatccgggacaaactaaggatttatgctcactgcacaaaccaccacaaccacaactacgttcgtgggtccattacactttttatcatcaacttgcatcgatcaagaaagaaaatcaagctggctgggactctcagagacaagctggttcaccagtacctgctgcagccctatgggcaggagggcctaaagtccaagtcagtgcaactgaatggccagcccttagtgatggtggacgacgggaccctcccagaattgaagccccgcccccttcgggccggccggacattggtcatccctccagtcaccatgggcttttttgtggtcaagaatgtcaatgctttggcctgccgctaccgataagctatcctcacactcatggctaccagtgggcctgctgggctgcttccactcctccactccagtagtatcctctgttttcagacatcctagcaaccagcccctgctgccccatcctgctggaatcaacacagacttgctctccaaagagactaaatgtcatagcgtgatcttagcctaggtaggccacatccatcccaaaggaaaatgtagacatcacctgtacctatataaggataaaggcatgtgtatagagcagaatgtttctcttcatgtgcactatgaaaacgagctgacagcacactcccaggagaaatgtttccagacaactccccatgatcctgtcacacagcattataaccacaaatccaaaccttagcctgctgctgctgctgccctcagaggaagatgaggaaggaaaaaaaactgggtggacctacaaaaacccatcctctcccaactccttcttctctgcctctttcttgctgctgccctgagttttttgacacatctctttccataggggagtaatgggtgtgtcagccctggcctgctgggagagctgtttatatgatttcccggctgatgtatgagcgtgcgcacctgggttcctcacagtggcatccatcactggcagttcttctgggaagcgggtgcttcaaaagtaaaattacaatcacactccaaaaaaaaaaaaaaa

SEQ ID NO: 2 - protein, 534 aa, Homo sapiens [sequence omitted]

SEQ ID NO: 3 - DNA, 24 bp, Artificial Sequence
  Description of Artificial Sequence PCR Primers
ggcaacatcacttcgaacaatgtc

SEQ ID NO: 4 - DNA, 23 bp, Artificial Sequence
  Description of Artificial Sequence PCR Primers
cgagccagccatcatgaatgatg

SEQ ID NO: 5 - DNA, 23 bp, Artificial Sequence
  Description of Artificial Sequence PCR Primers
gagaggaaaggttcccaggacag

SEQ ID NO: 6 - protein, 543 aa, Homo sapiens
MLLRSKPALPPPLMLLLLGPLGPLSPGALPRPAQAQDVVDLDFFTQEPLHLVSPSFLSVTIDANLATDPRFLILLGSPKLRTLARGLSPAYLRFGGTKTDFLIFDPKKESTFEERSYWQSQVNQDICKYGSIPPDVEEKLRLEWPYQEQLLLREHYQKKFKNSTYSRSSVDVLYTFANCSGLDLIFGLNALLRTADLQWNSSNAQLLLDYCSSKGYNISWELGNEPNSFLKKADIFINGSQLGEDFIQLHKLLRKSTFKNAKLYGPDVGQPRRKTAKMLKSFLKAGGEVIDSVTWHHYYLNGRTATKEDFLNPDVLDIFISSVQKVFQVVESTRPGKKVWLGETSSAYGGGAPLLSDTFAAGFMWLDKLGLSARMGIEVVMRQVFFGAGNYHLVDENFDPLPDYWLSLLFKKLVGTKVLMASVQGSKRRKLRVYLHCTNTDNPRYKEGDLTLYAINLHNVTKYLRLPYPFSNKQVDKYLLRPLGPHGLLSKSVQLNGLTLKMVDDQTLPPLMEKPLRPGSSLGLPAFSYSFFVIRNAKVAACI